SEQUENCE LISTING (25 sequences)

SEQ ID NO: 1 - protein, 965 aa, Homo sapiens
MAAHGKLRRERGLQPEYEAQVKVMRGQLSEQLHCLELQGELRRDLLLELAEFMRRRAEVELEYSRGLDKLAERFTSRSGRLGGSSREQQSFRKEPTLLSPLHCWAMLLEHTRQQSRESATLSEVLAGPLAQRLSHIAEDVGRIVKKSKDLVQQLQDELLEVVSELQTQGYLAWASLQTKKTYHVYHLESMNAETKLREAERQEEKRSGKSALPTTTAASATTTITIETELHRKGSLKKGGRLVEKRQAKFLEHKLKCTKARNEYLLSLASVNAAVSNYYLHDILDLMDCCDTGFHLALEQALRSYTAAESRTQTSHMQGLGSLEEALEALDPPGDKAKVLEVHAMAFCPPLRFDYQPHEGDEVAEIQVEMELRDEILPRAQNIQSRLDQKTIETEEVNKTLKATLQALLEVVASEDGDILDSLQASPSTESLKSTSSDPGTRQAGRRRNQQQETETFYITKLQEYLSGRSILAKLQAKHEKLQEAIQRGNKEEQETSSWTQCTERKFHKSHHPYPGSQCTQRLFGGDLEKFIQSTGQPVPLVVKSCIRFINLNGLQHEGIFAVSGAQARILEIRDAFERGEDPLVEGCTAHDLDSVAGVLKLYFRSLEPPLFPLDMFNELLASAELEVVGERVELVSHLLSRLPRPVLVVLRYLFTFLNHLAQYSDENMMDSYNLAVCFGPTLLPVPAGQDPVALQGRVNQLVQTLILQPAWVFPPTAMLPGPIYEKCMAPPSASCLGDGQLESLAGEPELELKTGTKAQEDDSEGVVEAVACFAYTGRTAQELTFQQGDTLRLYARASSDWWRGEHAGVRGLIPHKYITLSEGDEKQMTGSGLQAMGESLSHPESVLTLEFTNRLELGTPPEALGPSGHRRHCLVPTSPEQHVEMDKAVAQNMDSVFKELLGKAAVRQGHGLASTASPSLGTRNLKPLACSSFSKNKVFSRGPGAPVSPSVSHPQGPDSTRKPV

SEQ ID NO: 2 - protein, 12 aa, Homo sapiens
AAVLLPVLLAAP

SEQ ID NO: 3 - protein, 24 aa, Homo sapiens
HPIQIAAFLARIPPISSIGTCILK

SEQ ID NO: 4 - protein, 21 aa, Homo sapiens
KETWWETWWTEWSQPKKKRKV

SEQ ID NO: 5 - DNA, 36 bp, Homo sapiens
ctaggcggccgcatggcggcgcacgggaagttgcgg

SEQ ID NO: 6 - DNA, 34 bp, Homo sapiens
gctagaattcgactggcttgcgagttgaatctgg

SEQ ID NO: 7 - DNA, 29 bp, Homo sapiens
catagcggccgcatggaacgctttactag

SEQ ID NO: 8 - DNA, 34 bp, Homo sapiens; identical to SEQ ID NO: 6

SEQ ID NO: 9 - DNA, 36 bp, Homo sapiens; identical to SEQ ID NO: 5

SEQ ID NO: 10 - DNA, 29 bp, Homo sapiens
gctagaattcagcctccacaactccctcg

SEQ ID NO: 11 - DNA, 25 bp, Homo sapiens
agaaagatctatggcggcgcacggg

SEQ ID NO: 12 - DNA, 33 bp, Homo sapiens
ctagaattccgactggcttgcgagttgaatctg

SEQ ID NO: 13 - DNA, 25 bp, Homo sapiens
agaaagatctatggcggcgcacggg

SEQ ID NO: 14 - DNA, 28 bp, Homo sapiens
ggacgaattcagtatgttccagcagcat

SEQ ID NO: 15 - DNA, 40 bp, Homo sapiens
ctgcaacatgaaggcatcttcgcggtatcaggtgcccagg

SEQ ID NO: 16 - DNA, 29 bp, Homo sapiens
acaagttggctgaacgctttacctgtctc

SEQ ID NO: 17 - DNA, 29 bp, Homo sapiens
taaagcgttcagccaacttgtcctgtctc

SEQ ID NO: 18 - DNA, 29 bp, Homo sapiens
acaaattagcggagcgattcacctgtctc

SEQ ID NO: 19 - DNA, 29 bp, Homo sapiens
tgaatcgctccgctaatttgtcctgtctc

SEQ ID NO: 20 - DNA, 36 bp, Homo sapiens
gcagaattcctgcaggccaagcatgaaaagctccag

SEQ ID NO: 21 - DNA, 36 bp, Homo sapiens
cataagcttctagctctccaactggccatcccccag

SEQ ID NO: 22 - DNA, 29 bp, Homo sapiens
acaagttggctgaacgctttacctgtctc

SEQ ID NO: 23 - DNA, 29 bp, Homo sapiens
taaagcgttcagccaacttgtcctgtctc

SEQ ID NO: 24 - DNA, 29 bp, Homo sapiens
acaaattagcggagcgattcacctgtctc

SEQ ID NO: 25 - DNA, 29 bp, Homo sapiens
tgaatcgctccgctaatttgtcctgtctc

(19) United States
(12) Patent Application Publication
(10) Pub. No.: US 2010/0168010 A1
(43) Pub. Date: Jul. 1, 2010

(54) COMPOSITIONS AND METHODS OF MODULATING CELL GROWTH AND MOTILITY

(76) Inventors: Alfred Malouf, Orange Village, OH (US); Scott W. Young, Gaithersburg, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/516,000

(22) PCT Filed: Nov. 21, 2007
(86) PCT No.: PCT/US07/85350

§ 371 Date: Feb. 25, 2010

Related U.S. Application Data
(60) Provisional application No. 60/866,949, filed on Nov. 22, 2006.

(51) Int. Cl. A61K 38/17; A61K 38/00; A61K 31/7088; C12N 5/02

(52) U.S. Cl. 514/12; 514/2; 514/44 R; 435/375

(57) ABSTRACT

A method of modulating cell growth and motility in subject includes administering to a cell expressing GTPase an amount of an ARHGAP4 regulating agent effective to regulate the growth and motility of the cell.

### RELATED APPLICATIONS

This application claims priority from U.S. Provisional Application No. 60/866,949, filed Nov. 22, 2006, the subject matter, which is incorporated herein by reference.

### GOVERNMENT FUNDING

This invention was made with government support under Grant No. NS41383 awarded by The National Institutes of Health. The United States government has certain rights in the invention.

### FIELD OF THE INVENTION

The present invention relates to compositions and methods of modulating cell growth and motility and to peptides that can be used to regulate cell growth and motility

### BACKGROUND

Spinal Cord Injury (SCI) represents a significant social and economic problem as traumatic injury of the spinal cord often results in permanent functional impairment. The complexity of the biological responses resulting from SCI have made it difficult to find successful strategies to repair the damage and restore motor function. After a SCI, mature neurons do not regenerate damaged axons in a robust manner, and many actually retract from the site of injury. Additionally, the deposition of inhibitory molecules further diminishes the ability of damaged axons to regenerate through the SCI injury site of and beyond. Most of the deficits associated with spinal cord injury result from the loss of axons that are damaged in the central nervous system (CNS). Axons are effectively the primary transmission lines of the nervous system and in the form of bundles they help make up nerves.

Unlike axons from peripheral nerves or even embryonic axons in the CNS, mature CNS axons do not exhibit potential for robust regeneration unless they are cut near to the cell body (Fawcett (2002) Spinal Cord 40:615-623). Indeed, cut axons often retract from the lesion site and form retraction balls (Steward et al. (2003) J. Comp. Neur. 459:1-8). Therefore, long axon tracks in the spinal cord are unlikely to regenerate without some type of intervention. Thus far, the best results have been obtained using the neurotrophin NT-3, enhancing levels of cAMP and inhibition of RhoGTPase. RhoGTPases are especially interesting because they are potent regulators of the actin cytoskeleton and cell motility (Hall (1998) Science 279:509-514; van Aelst and D′Souza-Schorey (1997) Genes and Devo. 11:2295-2322). However, RhoGTPases regulate a number of cellular functions such as cell-cell and cell-matrix adhesion, membrane trafficking, transcription and cell proliferation, which raises some concerns that they could produce unwanted side effects.

Growing axons move through their environment via the growth cone, which is at the tip of the axon. The growth cone has a broad sheet like extension called lamellipodia which contain protrusions called filopodia. The filopodia extend from the leading edge of migrating cells. They contain actin filaments cross-linked into bundles by actin-binding proteins. Actin plays a major role in the mobility of the axonal growth and cell mobility in general, by forming microfilaments. These linear polymers of actin drive actoclampin end-tracking motors to propel cell crawling, ameboid movement, and changes in cell shape.

Intracellular actin cytoskeletal assembly and disassembly are tightly regulated by cell signaling mechanisms. The regulation of cell motility typically involves the regulation of actin filament assembly at the leading edge of motile cells. RhoA, Rac1 and Cdc42 are small GTPases that regulate the signaling pathways that control actin filament assembly and disassembly, and cell motility (Dickson (2001) Curr. Opin. Neurobiol. 11(1):103-110). RhoA typically inhibits motility while Rac1 and Cdc42 typically enhance motility. These GTPases act as molecular on/off switches that initiate a cascade of events that directly regulate actin filament dynamics. They are switched “on” when they bind GTP. RhoGAPs enhance the hydrolysis of GTP to GDP, which switches the GTPases to their “off” state, thereby inhibiting the downstream signaling that regulates actin filament dynamics and motility. This increase in GTP hydrolysis is dependent on a highly conserved arginine residue in the GAP domain that directly interacts with the active region of GTPases (Moon and Zheng (2003) J. Biol. Chem. 278:4151-4159).

### SUMMARY

The present invention relates to a method of modulating cell growth and motility in cells expressing GTPases by administering to the cells a pharmaceutical composition that includes a ARHGAP4 regulating agent. ARHGAP4 regulating agent can regulate expression or bioactivity of ARHGAP4 in a cell.

In one aspect of the invention, the ARHGAP4 regulating agent can include purified polypeptide that is a competitive inhibitor of ARHGAP4. The cell can be a nerve cell (e.g., neuron) that expresses ARHGAP4. A competitive inhibitor of ARHGAP4 can be administered to the nerve cells to promote growth and motility of the nerve cell. The competitive inhibitor of ARHGAP4 can include a GAP region loss of function mutation. The loss of function mutation can include the deletion or substitution of the arginine at the 562 position of wildtype ARHGAP4. In one example, the competitive inhibitor of ARHGAP4 can include the amino acid sequence of SEQ ID NO: 1.

In another aspect, the ARHGAP4 regulating agent can include an interfering RNA (e.g., short interfering RNA (siRNA)) that inhibits or reduces expression of ARHGAP4 in a cell Inhibition or reduction of expression of ARHGAP4 in the nerve cell can promote the nerve cells growth and motility.

In a further aspect, the ARHGAP4 regulating agent can include recombinant ARHGAP4 that is administered to a cell to inhibit cell growth and motility. Examples of cells that the ARHGAP4 can be administered to can include metastatic cells, such as metastatic cancer cells.

The present invention also relates to a method of treating a subject in which a spinal cord injury has occurred. The method includes administering to the subject an ARHGAP4 regulating agent to decrease the expression or bioactivity of ARHGAP4 in the targeted cells of the subject.

The present invention further relates to a purified polypeptide for promoting growth and motility of a nerve cell. The purified polypeptide includes a competitive inhibitor of ARHGAP4. In an aspect of the invention, the competitive inhibitor can have an amino acid sequence substantially similar to wild type ARHGAP4 but with a loss of function mutation of the GAP region. The competitive inhibitor can also have the amino acid sequence of wild type ARHGAP4 but with a deletion or substitution of the arginine at the 562 position of the wild type ARHGAP4. The competitive inhibitor can further have the amino acid sequence of SEQ ID NO: 1.

The present invention further relates to a method of promoting axonal growth in a nerve cell. The method includes administering to the nerve cell an ARHGAP4 regulating agent such that axonal growth occurs. The agent can regulate the expression or bioactivity of ARHGAP4 in the cell. In an aspect of the invention, the regulation of the expression or bioactivity of ARHGAP4 in the cell can include decreasing expression or bioactivity of ARHGAP4 in the cell. The ARHGAP4 regulating agent can include a competitive inhibitor of ARHGAP4 and/or an interfering RNA that inhibits expression of ARGAP4 in the cell.

In another aspect of the invention, the competitive inhibitor can have a GAP loss of function mutation. By way of example, the competitive inhibitor can have an amino acid sequence of SEQ ID NO:1.

Yet another aspect of the present invention relates to a method of treating a nerve cell injury in a subject. The method includes administering to a nerve cell of the subject an ARHGAP4 regulating agent. The agent can decrease the expression or bioactivity of ARHGAP4 in the subject. The ARHGAP4 regulating agent can include a competitive inhibitor of ARHGAP4. The competitive inhibitor can have a GAP loss of function mutation, such as an amino acid sequence of SEQ ID NO:1. The ARHGAP4 regulating agent can also include an interfering RNA that inhibits expression of ARGAP 4 in the cell.

A further aspect of the invention relates to a method of regulating cell motility, such as cell motility associated with metastatic cancer and wound healing. The method includes administering to the cell expressing a GTPase a therapeutically effective amount of an ARHGAP4 regulating agent. The agent can regulate the bioactivity or expression of ARHGAP4 in the cell. In one example, the cell can comprise a metastatic neoplastic cell and the ARHGAP4 regulating agent can decrease neoplastic cell motility. In another example, the cell can be a fibroblast cell, and the ARHGAP4 regulating agent can increase cell motility of the fibroblast cell.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 illustrates the structural domains of the ARHGAP4 protein. ARHGAP4 contains N-terminal FCH (Fps/Fes/Fer/CIP4 homology), coiled-coil (CC) and ARNEY domains. The GAP domain is centrally located and the C-terminal contains a SH3 (Src homology 3) domain.

FIG. 2 illustrates endogenous ARHGAP4 protein is present in the peripheral zone of NIH/3T3 cells and growth cones. NIH/3T3 cells (A-C) and growth cones from dissociated dentate granule cell cultures (D-F) were immunostained for endogenous ARHGAP4 using a rabbit antibody and a mouse monoclonal antibody against β-tubulin III to label axons and the central region of growth cones. Fluorescence was visualized using biotinylated secondary antibodies labeled with avidin-Alexa 594 (red) and avidin-Alexa 488 (green). Images were viewed using a Leica confocal laser scanning microscope. Yellow indicates regions of overlapping immunoreactivity. The leading edge of the NIH/3T3 cell and the distal tips of filopodia of dentate granule cells are indicated by arrows in these representative images (n=3). Calibration=5 μm.

FIG. 3 illustrates the ARHGAP4 N-terminus is required for normal protein localization in NIH/3T3 cells and axon terminals. (A,B) Localization of FLAG-tagged full length ARHGAP4 protein was compared to FLAG-tagged N-terminal and C-terminal truncation mutants in NIH/3T3 cells transiently transfected using Lipofectamine 2000 (Invitrogen). Anti-FLAG immunohistochemistry was used to label mutant proteins using a biotinylated secondary antibody and avidin-Alexa 488. (A) Fluorescence intensity was analyzed in a semi-quantitative fashion using NIH Image software. The ratio (R) of fluorescence intensity at the tip of the leading edge to the intensity 20 μm from the tip was calculated. (B) Results of the analysis are shown as mean±S.E.M., and ANOVA was performed. (C,D) Localization of ARHGAP4 in axons and growth cones. (C) The distribution of full-length ARHGAP4 and of 3 mutant proteins was quantified by dividing the pixel intensity of the respective tags at the growth cone by the pixel intensity at the axon 40 μm from the growth cone, similar to the method used for NIH/3T3 cells. (D) Results are shown as mean±S.E.M., and ANOVA was performed using SigmaStat 3.1 software.

FIG. 4 illustrates the effects of actin and microtubule destabilizing drugs on the localization of full-length ARHGAP4 (1-965-FLAG). Wound assay experiments were performed on NIH/3T3 cells that expressed full length FLAG-tagged ARHGAP4 (1-965-FLAG). Three hours after wounding, the cells were treated with vehicle alone (DMSO), nocodazole, or cytochalasin-D to disrupt MTs and actin filaments, respectively. Fluorescent images were merged with brightfield images (C, F, I) to show leading edge boundaries.

FIG. 5 illustrates the effects of actin and microtubule destabilizing drugs on the localization of the C-terminal truncation mutant ARHGAP4 (1-770-FLAG). Wound assay experiments were performed on NIH/3T3 cells that expressed C-terminal deletion mutant FLAG-tagged ARHGAP4 (1-770-FLAG). Three hours after wounding, the control cells were treated with vehicle alone (DMSO), nocodazole, or cytochalasin-D to disrupt MTs and actin filaments, respectively. Fluorescent images were merged with brightfield images (C, F, I) to show leading edge boundaries.

FIG. 6 illustrates the effects of mutant ARHGAP4 protein expression on MF axon outgrowth: Dentate explants were transfected using ExGen 500 to express ARHGAP4 proteins. Representative images obtained using a SPOT CCD camera attached to a Nikon Optiphot-2 fluorescence microscope are shown. Explants were transfected with the FLAG parent vector as control (A1); full-length ARHGAP4 (1-965)-FLAG (A2); the GAP loss of function mutant (R562A)-FLAG (A3); the C-terminal truncation mutant (1-770)-FLAG (A4); or the N-terminal truncation mutant (72-965)-FLAG (A5). MF axons were visualized using an anti-β-tubulin III antibody and nuclei of granule cells seen at the edge of the explant were stained blue using DAPI. Mossy fiber axon outgrowth was analyzed. Results are shown as mean±S.E.M., and ANOVA analysis was performed using SigmaStat 3.1 software (B). Astrocytes (immunopositive for GFAP) were visualized for outgrowth assays as described in Experimental methods (not shown). Results of astrocyte outgrowth assays are shown as mean±S.E.M., and ANOVA was performed (C).

FIG. 7 illustrates the expression of the R562A GAP activity mutant in NIH/3T3 cells results in increased cell motility. To assess the importance of GAP activity on cell motility, the (R562A)-EYFP mutant protein was expressed in NIH/3T3 cells. The parent EYFP, full-length (1-965)-EYFP, or (R562A)-EYFP vectors were transfected into NIH 3T3 cells using Lipofectamine 2000. Twenty-four hours after transfection, a scratch/wound was made in a confluent area of the culture plate and migration of transfected cells was assessed 2, 4 and 8 h after the wound. The migration of transfected cells was quantified by counting the number of EYFP-positive cells at the line that represents the leading edge of migrating cells in the wound (fastest cells), and dividing by the total number of EYFP-positive cells between the wound edges (panel A). All percentage values were normalized to control values (cells expressing EYFP alone) and the ratio for control cells is represented by the horizontal dashed line (R=1.0, panel B). When compared to control cells expressing EYFP vector alone, significantly fewer cells expressing the full-length protein (1-965)-EYFP are observed at the leading edge of migrating cells at 2, 4 and 8 h (panel B, black bars), while significantly more cells are observed at the leading edge if they express the (R562A)-EYFP mutant at 4 and 8 h (panel B, white bars) (p≦0.001). Importantly, there is a dramatic difference between the migration of cells expressing the full-length versus the R562A mutant proteins (p values shown in panel B). These observations suggest that the GAP function of ARHGAP4 is extremely important for regulation of cell motility. ANOVA analysis was performed using SigmaStat 3.1 software; n=3-5.

FIG. 8 illustrates knockdown of endogenous ARHGAP4 increased motility of NIH/3T3 cells: NIH/3T3 cells were co-transfected with the indicated siRNAs and an EYFP expression vector to identify transfected cells. (A) Cell motility was analyzed as described in FIG. 7, in cells transfected with Control siRNA or ARHGAP4 siRNA. Results are shown as mean±S.E.M. and statistical analysis was performed using SigmaStat software; n=3. (B) Western analysis (IB) was performed on cell lysates. Densitometric analysis of immunoreactive ARHGAP4 and actin bands was used to normalize endogenous ARHGAP4 expression, which corresponded to a 40% reduction in response to ARHGAP4 siRNA.

FIG. 9 illustrates viral expression vectors for shRNA produce robust knockdown of protein in neurons. DG explants from P4-9 rat hippocampus were transduced with lentivirus expression vectors that contained separate promoters for independently expressing EGFP and shRNA targeting hnRNP-U or a control, reverse sequence of the shRNA. Explants were analyzed 3 days after viral transduction. Fluorescence microscopic analysis showed that the vast majority of cells were EGFP-positive (A). Western analysis showed that hn-RNP-U expression was knocked down 73% (B). All values are shown as the mean+/−SEM and are the result of four independent experiments (21 DG explants/treatment). * p=0.042.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, “neuronal cell” or “cell of the nervous system” include both neurons and glial cells.

As used herein, “CNS neuron” refers to a neuron whose cell body is located in the central nervous system. The term is also meant to encompass neurons whose cell body was originally located in the central nervous system (e.g., endogenously located in the CNS), but which have been explanted and cultured ex vivo, as well as the progeny of such cells. Examples of such neurons are motor neurons, interneurons and sensory neurons including retinal ganglion cells, dorsal root ganglion cells and neurons of the spinal cord.

As used herein, “central nervous system” refers to any of the functional regions of the brain or spinal cord. This definition is used commonly in the art and is based, at least in part, on the common embryonic origin of the structures of the brain and spinal cord from the neural tube.

The “peripheral nervous system” can be distinguished from the central nervous system, at least in part, by its differing origin during embryogenesis. Cells of the peripheral nervous system are derived from the neural crest and include neurons and glia of the sensory, sympathetic and parasympathetic systems.

As used herein, “soma” refers to the cell body of a neuron.

As used herein, “axon” and “neurite” are used interchangeably to refer to the single outgrowth which extends from a neuron and which will ultimately migrate to innervate a target tissue. The tip of the axon is referred to as the “growth cone”. Axons extend from a neuron to a target tissue, and are capable of conducting impulses. In the literature, the term “axon” is often used to refer to the outgrowth from a cell in vivo, and the term “neurite” is often used to refer to the outgrowth from a cell in vitro, however, the terms are used interchangeably herein without regard to whether the cells are found in vivo or in vitro.

As used herein, “dendrite” refers to the fine extensions from a neuron soma which pick up electrical and chemical impulses. The number of dendrites found on a given neuron vary extensively and depend on the specific neuron. Typical neurons may have multiple dendrites, but only a single axon, and it is the axon that migrates in response to cues to innervate a target tissue.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences, which control transcription of the naturally occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

“Homology” and “identity” are used synonymously throughout and refer to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

The “non-human animals” of the invention include mammals such as rats, mice, rabbits, sheep, cats, dogs, cows, pigs, and non-human primates.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, “proliferating” and “proliferation” refer to cells undergoing mitosis.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agent, material, or composition comprising one or more agents as described herein which is effective for producing some desired effect in a subject.

The present invention relates to a method of modulating cell growth and motility by administering to a cell expressing a GTPase (e.g., RhoGTpase) a Rho GTPase activating protein 4 (ARHGAP4) regulating agent. The ARHGAP4 regulating agent can include polypeptides and nucleic acids that modulate (e.g., inhibit or promote) the expression or bioactivity of ARHGAP4 in a cell (e.g., nerve cell).

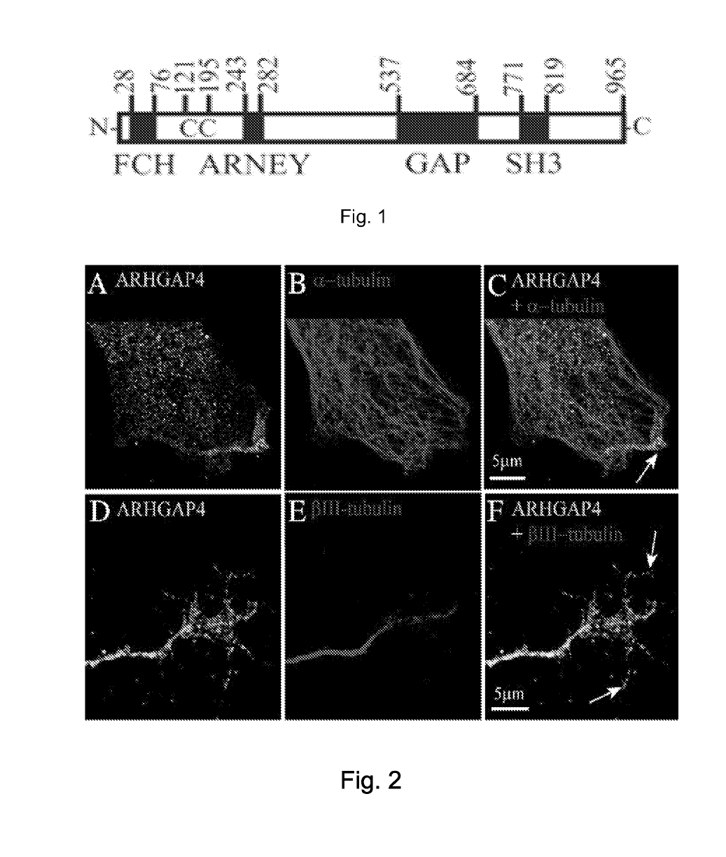

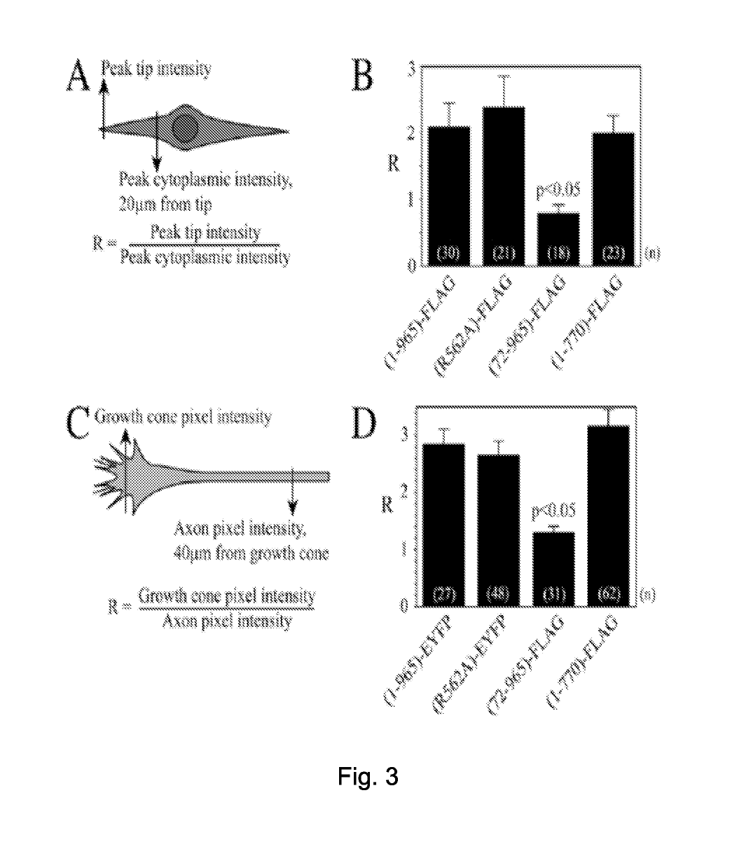

ARHGAP4 includes an N-terminal FCH (Fps/Fes/Fer/CIP4 homology) domain, a central GTPase activating (GAP) domain and a C-terminal SH3 (Src homology 3) domain (FIG. 1). It was found that ARHGAP4 inhibits cell migration and the outgrowth of hippocampal axons. The 1-71 fragment of the FCH domain of ARHGAP4 is responsible for its localization to the leading edge of NIH/3T3 cells and axons and growth cones (FIG. 3). The spatial targeting to the leading edge of migrating cells can contribute to ARHGAP4's regulation of motility. ARHGAP4 was also found to be a potent inhibitor of cell and axon motility, and its GAP activity plays a central role in mediating this inhibition. This inhibition of motility is consistent with the ability of ARHGAP4 to preferentially inhibit the function of Rac1 and Cdc42, which are RhoGTPases that are typically associated with enhancing axon outgrowth and cell motility. Agents that regulate ARHGAP4 (i.e., ARHGAP4 regulating agents) function can therefore be used to modulate (e.g., inhibit or promote) cell (e.g., nerve cell, neuronal cell, or axon) growth and motility.

One ARHGAP4 regulating agent in accordance with the present invention includes a purified polypeptide that is a dominant negative or competitive inhibitor of ARHGAP4. As used herein, “dominant negative” or “competitive inhibitor” refers to variant forms of a protein that inhibit the activity of the endogenous, wild type form of the protein (i.e., ARHGAP4). In the context of the present invention, a dominant negative or competitive inhibitor ARHGAP4 polypeptide is a ARHGAP4 polypeptide which has been modified (e.g., by mutation of one or more amino acid residues, by posttranscriptional modification, by posttranslational modification) such that the dominant negative ARHGAP4 inhibits the activity of the endogenous ARHGAP4.

In an aspect of the invention, the competitive inhibitor ARHGAP4 can be a purified polypeptide that has an amino acid sequence, which is substantially similar (i.e., at least about 75%, about 80%, about 85%, about 90%, about 95% similar) to the wild type ARHGAP4 but with a GAP region loss of function. By GAP region loss of function it is meant that wild type GAP is modified or deleted so that the ability of the ARGHAP4 to enhance the intrinsic GTPase activity of RhoGTPase, which turns off downstream signaling of these GTPases, is substantially inhibited. The purified polypeptide, which is a competitive inhibitor of ARHGAP4, can be administered to a cell expressing a GTPase (e.g., Pho GTPase), such as a nerve cell, to promote cell motility and/or axon growth.

In an aspect of the invention, the competitive inhibitor of ARHGAP4 can have an amino acid sequence of wild type ARHGAP4 except the arginine at the 562 position of the wild type protein is deleted or substituted with amino acid that results in GAP region loss of function. In one example, competitive inhibitor can include the amino acid sequence of SEQ ID NO: 1. The polypeptide of SEQ ID NO:1 is a modified or mutated ARHGAP4 where the arginine residue at position 562 of the wild type protein is replaced with an alanine residue (hereinafter the mutation is designated “(R562A)”).

It is shown in the examples below that the ARHGAP4 dominant negative protein is a potent enhancer of axon outgrowth as it leads to a decrease in ARHGAP4 function due to the (R562A) mutations dominant loss of function phenotype. A polypeptide comprising SEQ ID NO:1 can be utilized as an ARHGAP4 regulating agent to reduce the bioactivity of endogenous ARHGAP4. As used herein, “bioactivity” is defined as any activity having an effect upon a living organism, tissue, or cell such as the ability to enhance GTPase activity.

The competitive inhibitor of ARHGAP4 can also have other amino acid sequences that are substantially similar to ARHGAP4 as long as the resultant polypeptide retains a GAP region loss of function. For example, other competitive inhibitors can have an amino acid sequence of SEQ ID NO:1 but with amino acid residue insertions at the 562 positions, deletion of the arginine residue at the 562 position or other deletions and substitutions that result in GAP region loss of function but still allows the polypeptide to be a competitive inhibitor of ARHGAP4.

The competitive inhibitor polypeptides of the present invention can also be modified by natural processes, such as posttranslational processing, and/or by chemical modification techniques, which are known in the art. Modifications may occur anywhere in the polypeptide including the polypeptide backbone, the amino acid side-chains and the amino or carboxy termini. It will be appreciated that the same type of modification may be present in the same or varying degrees at several sites in a given polypeptide. The polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular proterties, 2^{nd} Ed., T. E. Creighton, W.H. Freeman and Company, New-York, 1993).

Other type of polypeptide modification may include for example, amino acid insertion (i.e., addition), deletion and substitution (i.e., replacement), either conservative or non-conservative (e.g., D-amino acids) in the polypeptide sequence where such changes do not substantially alter the overall bioactivity (e.g. GAP loss of function) of the polypeptide.

Polypeptide competitive inhibitors of the present invention may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may promote axonal growth (without being restricted to the present examples).

The polypeptide competitive inhibitors of the present invention may be prepared by methods known to those skilled in the art. The polypeptides may be prepared using recombinant DNA. For example, one preparation can include cultivating a host cell (bacterial or eukaryotic) under conditions which provide for the expression of polypeptides within the cell. More specifically, a mammalian cell line (e.g. a CHO modified mammalian cell line) can be used to ensure proper folding and post-translational modification of the resultant recombinant polypeptide.

The purification of the polypeptides may be done by affinity methods, ion exchange chromatography, size exclusion chromatography, hydrophobicity or any other purification technique typically used for protein purification. The purification step can be performed under non-denaturating conditions. On the other hand, if a denaturating step is required, the protein may be renatured using techniques known in the art.

The competitive inhibitor polypeptides can be provided in a pharmaceutical composition. The pharmaceutical compositions can include an effective amount of the purified polypeptides as described above (e.g. those including the amino acid sequence of SEQ ID NO: 1) and a pharmaceutically acceptable diluent or carrier.

The term “pharmaceutically acceptable carrier” “diluents”, or “adjuvant” and “physiologically acceptable vehicle” and the like are to be understood as referring to an acceptable carrier or adjuvant that may be administered to a patient, together with an ARHGAP4 regulating agent of this invention, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

In addition, the term “pharmaceutically effective amount” or “therapeutically effective amount” refers to an amount (dose) effective in treating a patient, having, for example, a nerve injury or a state characterized by diminished potential for axonal growth. It is also to be understood herein that a “pharmaceutically effective amount” may be interpreted as an amount giving a desired therapeutic effect, either taken into one dose or in any dosage or route or taken alone or in combination with other therapeutic agents. In the case of the present invention, a “pharmaceutically effective amount” may be understood as an amount of ARHGAP4 regulating agent to reduce or increase the bioactivity of ARHGAP4, to reduce the expression of ARHAGP4, to promote axonal growth, or to reduce or promote cell motility.

Determination of a therapeutically effective amount is within the capability of those skilled in the art. The exact formulation, route of administration and dosage can be chosen by the individual physician in view of the patient's condition.

The polypeptide of the present invention can also be in the form of a conjugate protein or drug delivery construct having at least a transport subdomain(s) or moiety(ies) (i.e., transport moieties). The transport moieties can facilitate uptake of the ARHGAP4 regulating polypeptide into a mammalian (i.e., human or animal) tissue or cell. The transport moieties can be covalently linked to ARHGAP4 regulating polypeptide. The covalent link can include a peptide bond or a labile bond (e.g., a bond readily cleavable or subject to chemical change in the interior target cell environment). Additionally, the transport moieties can be cross-linked (e.g., chemically cross-linked, UV cross-linked) to the polypeptide.

The transport moieties can be repeated more than once in the polypeptide. The repetition of a transport moiety may affect (e.g., increase) the uptake of the ARHGAP4 regulating agent by a desired cell. The transport moiety may also be located either at the amino-terminal region of an active agent or at its carboxy-terminal region or at both regions.

In an aspect of the invention, the transport moiety can include at least one transport peptide sequence that allows the ARHGAP4 regulating polypeptides to penetrate into the cell. Examples of transport sequences that can be used in accordance with the present invention include a Tat-mediated protein delivery sequence (Vives (1997) 272: 16010-16017), polyargine sequences (Wender et al. 2000, PNAS 24: 13003-13008) and antennapedia (Derossi (1996) J. Biol. Chem. 271: 18188-18193). Other examples of known transport moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, (conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No.: 2,301,157 (Crisanti et al,) incorporated by reference in their entirety). Similarly, HIV Tat protein was shown to be able to cross cellular membranes (Frankel A. D. et al., Cell, 55: 1189).

In addition, the transport moiety(ies) can include polypeptides having a basic amino acid rich region covalently linked to an active agent moiety ARHGAP4 regulating polypeptide. As used herein, the term “basic amino acid rich region” relates to a region of a protein with a high content of the basic amino acids such as arginine, histidine, asparagine, glutamine, lysine. A “basic amino acid rich region” may have, for example 15% or more (up to 100%) of basic amino acids. In some instance, a “basic amino acid rich region” may have less than 15% of basic amino acids and still function as a transport agent region. More preferably, a basic amino acid region will have 30% or more (up to 100%) of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of the present invention can function as a transport agent region.

Other transport sequences that have been tested in other contexts, (i.e., to show that they work through the use of reporter sequences), are known. One transport peptide, AAVLLPVLLAAP (SEQ ID NO:2), is rich in proline. This transport made as a GST-MTS fusion protein and is derived from the h region of the Kaposi FGF signal sequence (Royas et al. (1998) Nature Biotech. 16: 370-375). Another example is the sperm fertiline alpha peptide, HPIQIAAFLARIPPISSIGTCILK (SEQ ID NO: 3) (See Pecheur, J. Sainte-Marie, A. Bienvenuje, D. Hoekstra. 1999. J. Membrane Biol. 167: 1-17).

In one example, the ARHGAP4 regulating polypeptide can be provided as a fusion protein (polypeptide) that includes a carboxy terminal ARHGAP4 regulating polypeptide moiety and an amino terminal transport moiety. The amino terminal transport moiety can be a transport subdomain of HIV (e.g., HIV-1) Tat protein, homeoprotein transport sequence, a Histidine tag or a functional derivative and analogues thereof (i.e. pharmaceutically acceptable chemical equivalents thereof). In another example, the fusion protein (polypeptide) can include a carboxy terminal ARHGAP4 moiety and an amino terminal transport moiety that includes a homeodomain of antennapedia.

In another aspect of the invention, the ARHGAP4 regulating polypeptide can be non-covalently linked to a transfection agent. An example of a non-covalently linked polypeptide transfection agent is the Chariot protein delivery system (See U.S. Pat. No. 6,841,535; Morris et al. (1999) J. Biol. Chem. 274(35):24941-24946; and Morris et al. (2001) Nature Biotech. 19:1173-1176), all herein incorporated by reference in their entirety.

The Chariot protein delivery system includes a peptide transfection agent that can non-covalently complex with the ARHGAP4 regulating polypeptide of the present invention. Upon cellular internalization, the transfection agent dissociates and the ARHGAP4 regulating agent is free to function. In one example, the Chariot transfection agent can have the amino acid sequence: KETWWETWWTEWSQPKKKRKV-cya (cysteamine) (SEQ ID NO: 4). The complex of the Chariot transfenction peptide and the ARHGAP4 regulating peptide can be delivered to and internalized by mammalian cells allowing for higher dosages of therapeutics to be delivered to the site of pathology. A molar excess of peptide transfection agent relative to the ARHGAP4 regulating peptide to be delivered can be employed to accomplish peptide transfection.

The ARHGAP4 regulating agent of the present invention can also include an agent that reduces or inhibits ARHGAP4 expression in nerve cells to promote nerve cell growth and motility (e.g., axon growth and motility). In one example, the agent can be an antisense oligonucleotide. Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein (e.g., ARHGAP4).

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups, such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an anomeric oligonucleotide. An anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

ARHGAP4 regulating agent can also include a RNAi construct that inhibits or reduces expression of ARGHAP4. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAI construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

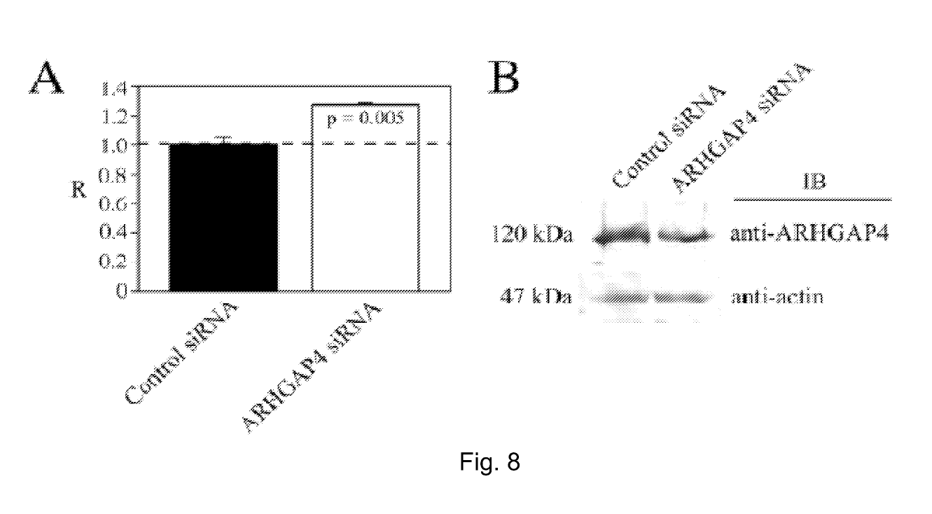

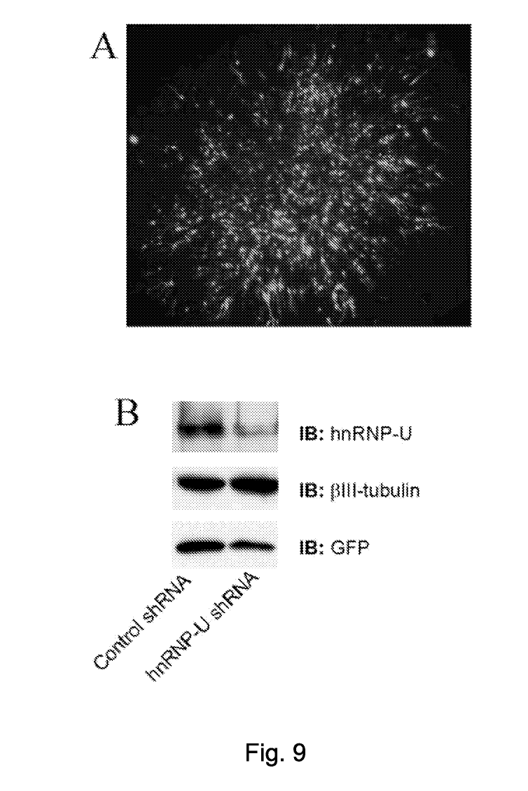

By way of example, a lentiviral vector can be used for the long-term expression of a siRNA, such as a short-hairpin RNA (shRNA), to knockdown expression of ARHGAP4 in a nerve cell. Although there have been some safety concerns about the use of lentiviral vectors for gene therapy, self-inactivating lentiviral vectors are considered good candidates for gene therapy (FIG. 9) as they readily transfect nerve cells. Alternatively, commercially available siRNA against ARHGAP4 can be used as an ARHGAP4 regulating agent in the methods of the present invention (FIG. 8).

The ARHGAP4 regulating agent of the present invention can be administered to a cell (e.g., nerve cell) of a subject by contacting the cell of the subject with a pharmaceutical composition comprising the ARHGAP4 regulating agent. In one aspect, a pharmaceutical composition comprising the ARGHGAP4 regulating agent can be administered directly to the cell (e.g., nerve cell adjacent a spinal cord injury) by direct injection or intrathecal injection. Alternatively, cells may be isolated from a mammal and treated (exposed) ex-vivo (e.g., in gene therapy techniques) with the delivery agent of the present invention before being re-infused in the same individual or in a compatible individual.

In a further aspect of the invention, the ARHGAP4 regulating agents can be used in combination and adjunctive therapies for modulating cell growth and motility, such as in therapies for promoting axon growth following spinal cord injury.

The phrase “combination therapy” embraces the administration of ARHGAP4 regulating agent, and a therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical. “Combination therapy” also can embrace the administration of the therapeutic agents as described above in further combination with other biologically active ingredients (such as, but not limited to, a second and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrase “adjunctive therapy” encompasses treatment of a subject with agents that reduce or avoid side effects associated with the combination therapy of the present invention.

In an aspect of the invention, the therapeutic agent administered in combination with the ARHGAP4 regulating agent can include an extracellular axon outgrowth inhibitory molecules derived from myelin (e.g., Nogo, MAG) and astrocytes (e.g., CSPGs). For example, the additional agents can include a Nogo inhibitor (e.g. an antibody against Nogo). Nogo has a 66-residue lumenal/extracellular domain which inhibits axonal extension and collapses dorsal root ganglion growth cones (GrandPre et al (2000) Nature 403:439-444). Another agent which can be administered in ARHGAP4 regulating agent is chondroitinase ABC for enzymatic inactivation of CSPGs. It has been shown that intrathecal treatment with chondroitinase ABC upregulated a regeneration-associated protein in injured neurons, and promoted regeneration of corticospinal tract axons. (Bradbury et al. (2002) Nature 416, 636-640).

The ARHGAP4 regulating agent can also be administered in conjunction with a RhoGTPase inhibitor to treat a nervous system injury, such as a spinal cord injury. The RhoGTPase inhibitor may include a RhoA inhibitor, a Rac1 inhibitor, or a Cdc42 inhibitor. It is also contemplated that combinations of additional RhoGTPase inhibitors may be administered to a subject having a spinal cord injury. A particular Rho A inhibitor useful in the methods of the present invention is ADP-ribosyl transferase C3 or as pharmaceutically composed, Cethrin. Cethrin may be particularly useful as it is derived from C3, which acts primarily on RhoA, while ARHGAP4 acts primarily on Rac and Cdc42. Cethrin is known to reduce the damage from spinal cord injury and stimulates axon regeneration when applied to the spinal cord. The Rho family GTPases are known in the art to regulate axon growth and regeneration. Inactivation of Rho with C3, a toxin from Clostridium botulinum (see Saito et al., 1995, FEBS Lett 371:105-109; Wilde et al., 2000. J. Biol. Chem. 275:16478), can stimulate regeneration and sprouting of injured axons. Cethrin is a recombinant protein drug which acts as a Rho A antagonist to promote neuroregeneration and neuroprotection in the Central Nervous System (CNS). C3 is known to be fairly nontoxic to cells (Dillon and Feig (1995) Meth. Enzy. 256:174-184).

Cethrin is pharmaceutically composed to effectively penetrate into CNS tissue, where it has been clearly shown to elicit the rescue and repair of damaged neurons. Thus, in another aspect of the present invention ADP-ribosyl transferase C3 or fragments thereof retaining ADP-ribosyl transferase activity can be administered to the subject in addition to an ARHGAP4 regulating agent. Cethrin is typically delivered in a single dose directly onto the dura mater of the spinal cord during decompression/stabilization surgery.

The ARGHGAP4 regulating agents can be used to treat CNS injuries, such as a spinal cord injury. Trauma to the spinal cord can result from contusion, compression or laceration injuries. Since most of the deficits associated with spinal cord injury result from the loss of axons that are damaged in the central nervous system, the present invention also relates to methods of treating a subject that has suffered a spinal cord injury. The methods contemplated in the present invention include administering an ARHGAP4 regulating agent to the subject and allowing for the agent to decrease the expression or bioactivity of ARHGAP4 in the subject. In one particular example, the ARHAGP4 regulating agent can be administered directly to a central or peripheral nervous system lesion site, such as by direct injection and/or intrathecal injection.

The ARGHGAP4 regulating agent can also be used to treat other diseases of the CNS that are associated with axonal loss and retraction, such as stroke, human immunodeficiency virus (HIV) dementia, prion diseases, Parkinson's disease, Alzheimer's disease, multiple sclerosis and glaucoma. Common to all of these diseases is the loss of axonal connections with their targets, and cell death. The ability to promote and stimulate growth of axons from the affected or diseased neuronal population would improve recovery of lost neurological functions, and protection from cell death can limit the extent of damage. For example, following a white matter stroke, axons are damaged and lost, even though the neuronal cell bodies are alive, and stroke in grey matter kills many neurons and non-neuronal (glial) cells. Treatments that are effective in eliciting sprouting from injured axons can be equally effective in treating some types of stroke. Neuroprotective agents often tested as potential compounds that can limit damage after stroke. Compounds which show both growth-promotion and neuroprotection are especially good candidates for treatment of stroke and neurodegenerative diseases. Similarly, although the present invention will generally relate to delivery of ARHGAP4 regulating agents, etc. to a traumatically damaged nervous system, this invention may also be applied to a method of treating a subject for a state characterized by diminished potential for axonal growth, such as (but not limited to) during stroke, multiple sclerosis, HIV dementia, Parkinson's disease, Alzheimer's disease, amyotrophic lateral sclerosis (ALS), prion diseases or other diseases of the CNS were axons are damaged in the CNS environment.

Treatment with ARHGAP4 regulating agents can also be used to enhance the rate of axon growth of peripheral nerves and thereby be effective for repair of peripheral nerves after surgery, for example after reattaching severed limbs. Also, treatment with polypeptides and pharmaceutical compositions of the present invention are expected to be effective for the treatment of various peripheral neuropathies because of their axon growth promoting effects.

Another aspect of the present invention relates to methods of regulating cell motility. The method includes administering to a cell a therapeutically effective amount of an ARHGAP4 regulating agent, such as ARHGAP4 protein or a mimetic thereof, to decrease cell motility. It may be beneficial to decrease or inhibit the cell motility of metastatic neoplastic cells. This may be especially useful in the treatment of neoplastic diseases such as cancers as Rho is an important target for treatment of cancer and metastasis (Clark et al (2000) Nature 406:532-535).

An ARHGAP4 regulating agent can also include an ARHGAP4 competitive inhibitor in order to promote cell motility. More specifically the ARHGAP4 regulating agent can include a polypeptide containing amino acid SEQ ID NO:1 to promote cell motility. The methods of the present invention may be beneficial to promote fibroblast cell motility as fibroblasts provide a structural framework for many tissues, and play a critical role in wound healing.

### Example 1

### Endogenous ARHGAP4 is Localized to the Leading Edge of Migrating NIH/3T3 Cells and to Axons and Growth Cones

Endogenous ARHGAP4 has been shown to be expressed in NIH/3T3 fibroblasts, NRK epithelial cells and PC12 cells. These data showed ARHGAP4 was localized to the golgi, along microtubules in NRK cells, and at the tips of extending neurites of NGF-treated (neuronally differentiated) PC12 cells. Our data showed that ARHGAP4 was also localized to the leading edge of migrating NIH/3T3 cell fibroblasts (FIGS. 2A-C). Mossy fiber (MF) growth cones from dissociated dentate granule cell cultures were immunostained for ARHGAP4 and costained using an antibody against β-tubulin III (FIGS. 2D-F). These data show that ARHGAP4 is localized to axons and growth cones, including the tips of filopodia.

### The FCH Domain is Important for Localizing ARHGAP4 to Growth Cones and to the Leading Edge of NIH/3T3 Cells

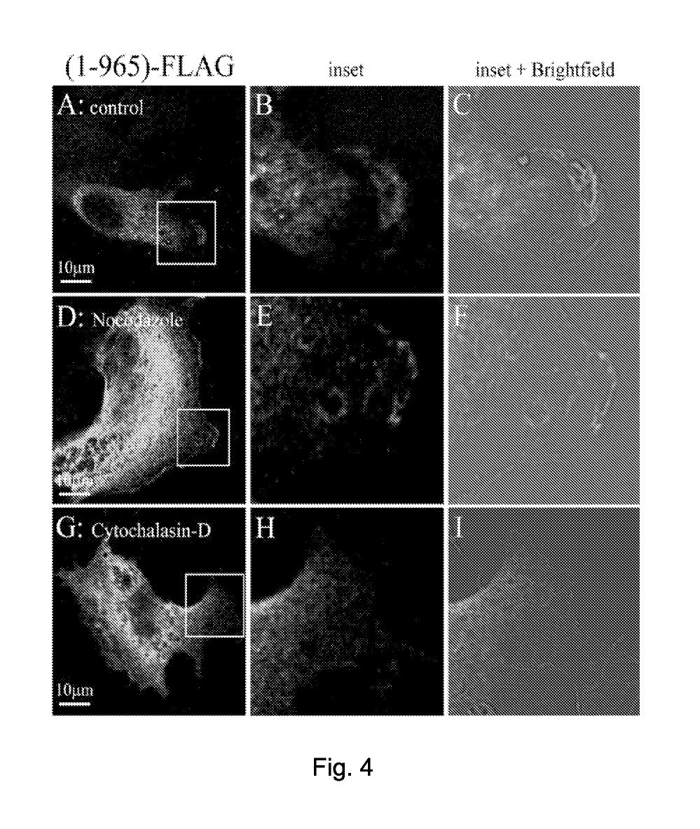

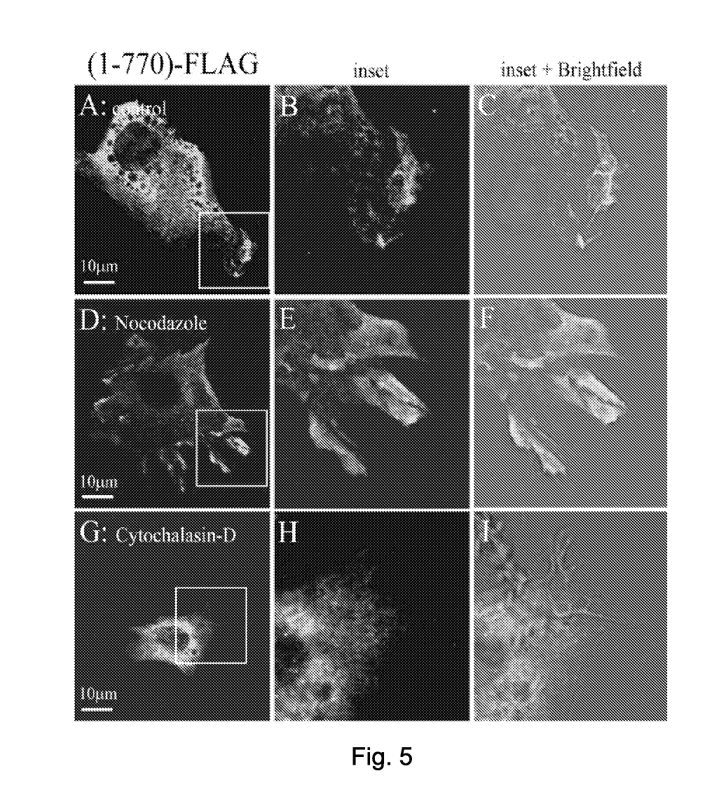

Structural and functional analyses were performed to identify the role of the different domains in targeting ARHGAP4 to the extreme peripheral tips of NIH/3T3 cells (FIGS. 3A, B). Western analysis confirmed that our transfection methods resulted in the expression of FLAG-tagged proteins of appropriate size. Fluorescent images of full-length protein (1-965)-FLAG and the C-terminal truncation (1-770)-FLAG are shown in FIGS. 4 and 5. The wealth of knowledge about the regulation of MT and actin cytoskeletal dynamics in NIH/3T3 cells during motility makes them a valuable model for the study of RhoGAP function. For these studies, the location of a full-length ARHGAP4 FLAG-tagged fusion protein was compared to that of FLAG-tagged N-terminal and C-terminal ARHGAP4 truncation mutants. Fluorescence intensity was analyzed in a semi-quantitative fashion to calculate the ratio of fluorescence intensity at the peripheral tip of the cell compared to the intensity 20 μm from the tip (FIG. 3A). These data show that the ARHGAP4 N-terminal truncation mutant (72-965)-FLAG was less specifically localized to the peripheral region of NIH/3T3 cells than the full-length protein (1-965)-FLAG, the C-terminal truncation mutant (1-770)-FLAG, or the GAP loss-of-function (GAP LOF) mutant (R562A)-FLAG (FIG. 3B). The same proteins were expressed in dissociated cultured granule cell neurons to identify regions responsible for targeting ARHGAP4 to axons and the peripheral region of growth cones and the data were quantified (FIGS. 3C, D). As in NIH/3T3 cells, only the N-terminal truncation mutant, (72-965)-FLAG, exhibited a reduced localization in growth cones compared to full-length ARHGAP4 (fluorescent images not shown). These data suggest that the N-terminal FCH domain is necessary for localizing ARHGAP4 to the tips of both NIH/3T3 cells and axon growth cones, but that neither a functional GAP domain nor the C-terminal domain was important for this localization. The cytoskeleton and ARHGAP4 localization the role of MTs and actin filaments in the localization of ARHGAP4 to the leading edge of NIH3T3 cells was examined. Nocodazole and cytochalasin-D were used to destabilize MTs and actin filaments, respectively. NIH/3T3 cells were transiently transfected to express FLAG-tagged full length ARHGAP4 (FIG. 4), the C-terminal truncation mutant 1-770 (FIG. 5) or the N-terminal truncation mutant 72-965 (data not shown). Forty-eight hours after transfection, a confluent region in the culture was scratched/wounded to induce polarization of the cytoskeleton and directed movement of the NIH/3T3 cells towards the center of the wound. Three hours after wounding, the cells were treated with either 300 nM nocodazole or 1 μM cytochalasin-D, which disrupted the normal pattern of microtubules and actin filaments in these cells. The cells were fixed 5 min later and the distribution of ARHGAP4 expression proteins were analyzed. The results show that full-length ARHGAP4 accumulates at the periphery of the leading edge of migrating NIH/3T3 cells (FIGS. 4A-C), and that this localization is disrupted when actin filaments are destabilized (FIGS. 4G-I) but not when MTs are destabilized (FIGS. 4D-F). A similar result was observed for the 1-770 C-terminal truncation mutant protein (FIG. 5). However, as previously described in FIG. 3, the 72-965 N-terminal truncation mutant was not localized to the leading edge of migrating NIH/3T3 cells and this pattern was not altered by either nocodazole or cytochalasinD (data not shown). These observations suggest that amino acids 1-71 are necessary for transport of ARHGAP4 to the cell periphery, and that localization of ARHGAP4 at the cell cortex appears to be dependent on its interaction with actin filaments.

### ARHGAP4 Inhibits Axon Outgrowth

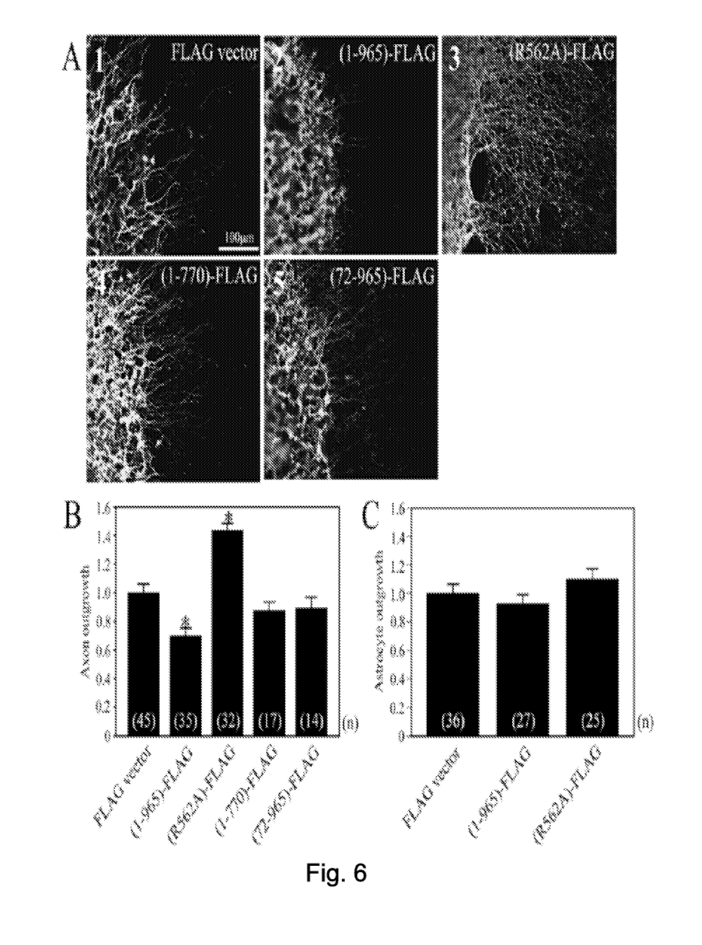

The function of ARHGAP4 on axon outgrowth was examined (FIG. 6). To determine whether or not ARHGAP4 regulates axon outgrowth, control dentate explants were transfected with FLAG empty vector (FIG. 6A1), or the ARHGAP4 proteins: full-length (1-965)-FLAG (FIG. 6A2), the R562A GAP-LOF mutant (FIG. 6A3), the C-terminal truncation mutant (1-770)-FLAG (FIG. 6A4), or the N-terminal truncation mutant (72-965)-FLAG (FIG. 6A5). Western analysis demonstrated the expression of EYFP-tagged proteins in homogenized extracts of dentate explants, and immunofluorescence images showed that these proteins are strongly expressed in somata located in the granule cell layer of the explants. MF axon and astrocyte outgrowth was measured as previously described (Butler et al. (2004) J. Neurosci. 24:462-473) and outgrowth data were quantified in FIGS. 6B and C. Only expression of the full-length (1-965)-FLAG protein inhibited MF outgrowth (FIG. 6B), suggesting that both the N- and C-terminal regions of ARHGAP4 are important for regulating axon outgrowth. Similar results were observed in dissociated hippocampal neurons. ARHGAP4 specifically altered axon but not astrocyte migration from dentate explants (FIGS. 6B, C). In view of the fact that only N-terminal truncation of ARHGAP4 altered its localization to the periphery of axons and fibroblasts, the N-terminal truncation data in FIG. 6 suggest that ARHGAP4 must be properly localized in the growth cone to inhibit axon outgrowth. Results obtained with the C-terminal truncation mutant also show that even when ARHGAP4 is properly localized, the C-terminal region is necessary for inhibition of axon outgrowth, and suggest that ARHGAP4's function is dependent on important interactions between its C-terminus and other proteins in growth cones.

### The GAP Function of ARHGAP4 is a Potent Regulator of Axon Outgrowth

ARHGAP4 has been shown to enhance GTP hydrolysis by Rho GTPases. However, it is unknown if its GAP function is important for the regulation of axon outgrowth or cell migration. In order to assess the importance of the GAP domain in axon outgrowth, the R562A mutant protein was expressed in granule cells. Residue 562 is the essential arginine in the arginine finger region of ARHGAP4. The standard method for examination of GAP domain function is to mutate this essential arginine to an alanine to generate a GAP loss of function (LOF) mutant. Although an R to A substitution mutant exhibits dramatically reduced GAP activity, it should retain all of the binding interactions for the unchanged FCH and SH3 regions. Because RhoGAPs with essential arginine mutations still bind their effector proteins, their lack of GAP activity can enable these mutant proteins to have a dominant-negative function. Indeed, the (R562A)-FLAG mutant protein is appropriately targeted to the peripheral tip of axons and NIH/3T3 cells (FIG. 3). In contrast to the inhibitory effects of full-length ARHGAP4, MF explants and dissociated neurons expressing the (R562A)-FLAG protein showed significantly greater outgrowth (FIGS. 6A3, B). Interestingly, MF outgrowth in (R562A)-expressing granule cells was not only longer, but appeared to be more branched and/or defasciculated (FIG. 6A3). These observations suggest that the GAP function of ARHGAP4 regulates several aspects of axon behavior.

### ARHGAP4 Inhibited Cell Motility and the GAP-LOF Mutant Enhanced Motility

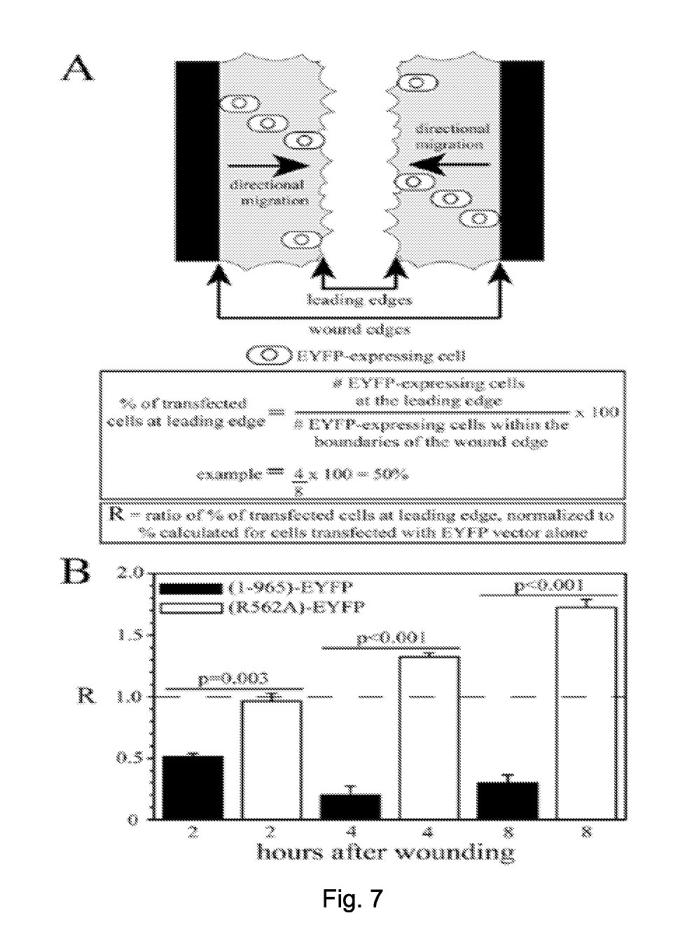

Since cytoskeletal elements must become organized in a polarized fashion to enable the directional migration of cells, the in vitro wound/scratch assay has become a useful method for studying the function of proteins that are involved in organizing the actin and MT cytoskeleton. Therefore, the wound assay was used to examine the effect of ARHGAP4 overexpression on NIH/3T3 cell migration. NIH/3T3 cells were transfected to express full-length EYFP-tagged ARHGAP4, and migration was quantified as described in FIG. 7A and Experimental methods. When compared to control cells that expressed the EYFP vector alone (dashed line), a significantly smaller percentage of cells that expressed the full-length protein (1-965)-EYFP was observed at the forward edge of migrating cells at 2, 4 and 8 h (FIG. 7B, black bars) (p≦0.001).

Cells expressing the (R562A)-EYFP mutant represented a significantly higher percentage of cells at the forward edge at 4 and 8 h (FIG. 7B, white bars) (p≦0.001). Importantly, there was a dramatic difference between the migration of cells that expressed full-length versus R562A mutant proteins (p values shown in panel B), suggesting that ARHGAP4 inhibits cell motility. The fact that the (R562A)-EYFP expressing cells migrate faster than control cells (dashed line) also suggests that this mutant protein acts as a dominant-negative mutation. Reducing the level of endogenous ARHGAP4 by 40% using siRNA also enhanced cell motility (FIG. 8). These data provide strong evidence that endogenous ARHGAP4 functions as a potent inhibitor of NIH/3T3 cell migration.

### Lentiviral Vectors

Our previous experience has demonstrated that lentiviral vectors expressing short-hairpin RNA (shRNA) are capable of knocking down protein expression in dentate explants. These experiments focused on the mRNA binding protein hnRNP-U. Although the shRNA sequence was derived from an siRNA sequence that produced a non-significant reduction in protein expression, lentiviral expression of shRNA produced a 73% reduction in protein expression (FIG. 9). The difference between the results obtained using siRNA and shRNA are probably due to our inability to deliver high concentrations of siRNA into cells and to the lower percentage of cells taking up the siRNA relative to the high efficiency and expression levels produced by lentiviral transformation of these neurons.

### Discussion

This investigation examined the structure and function of ARHGAP4, a novel RhoGAP. RhoGAPs enhance the intrinsic GTPase activity of RhoGTPase, which turns off downstream signaling of these GTPases. The well-defined role of RhoGTPases in regulating actin dynamics suggests that RhoGAPs provide an important link in the pathways that control cellular movement. RhoGAPs localized to the leading edge of migrating cells and axonal growth cones are in the proper location to regulate cytoskeletal dynamics that control movement, and our study shows that ARHGAP4 is strongly localized to axons and growth cones in neurons and to the peripheral tips of NIH/3T3 cells (FIGS. 2-4). ARHGAP4 appears to play a central role in the inhibition of both NIH/3T3 cell motility and axon outgrowth as demonstrated by the overexpression of full-length ARHGAP4. This inhibitory function is further supported by the enhancement of motility following a knockdown of endogenous ARHGAP4 expression using siRNA or endogenous ARHGAP4 function by expression of the dominant negative R562A mutant protein. The significant enhancement of outgrowth produced by the dominant-negative R562A mutant protein also suggests that the GAP domain of ARHGAP4 plays an important role in its regulation of motility. This inhibition of motility is consistent with the ability of ARHGAP4 to inhibit the function of Rac1 and Cdc42, which are RhoGTPases that are typically associated with enhancing axon outgrowth and cell motility (Govek et al. (2005) Genes Dev. 19:1-49; Negishi and Katoh, (2002) J. Biochem 132:157-166). Truncated ARHGAP4 72-965 does not appear to be efficiently transported to the distal end of axons and growth cones or to the peripheral region of NIH/3T3 cells. These data show that the N terminal FCH domain is critical for localizing ARHGAP4 to the leading edge of migrating NIH/3T3 cells and axon growth cones (FIG. 3). ARHGAP4 72-965 also loses its ability to regulate axon motility (FIG. 6), suggesting that ARHGAP4 must be properly localized at the leading edge to affect motility. Accumulation at the leading edge appears to involve interactions with actin filaments rather than MTs (FIGS. 4 and 5). When motile NIH/3T3 cells were treated with nocodazole to destabilize MTs, ARGAHP4 remained localized to the cell cortex (FIGS. 4D-F), while destabilizing actin filaments with cytochalasin-D abolished its localization to the cortex (FIGS. 4G-I). Unfortunately, we were not able to identify whether the N- or C-terminal regions of ARHGAP4 mediated this actin binding. This was because the N-terminal deletion mutant (72-965) does not traffic to the cell periphery, and although the 1-770 C-terminal deletion fragment appears to be localized to the periphery (FIG. 5C) it does not appear to be aligned as neatly with the cell cortex as the full-length protein (FIG. 4C). These observations are consistent with recently published data showing that the C-terminus SH3 domain of ARHGAP4 binds to the scaffolding protein Hem-1 (Weiner et al. (2006)). Weiner et al. showed that Hem-1 generated a complex of proteins that is targeted to the leading edge of polarized neutrophils. Several of these proteins, included Abi-1, Nck and ARHGAP4, appear to interact with Hem-1 through their homologous SH3 domains. The authors suggest that the Hem-1 leading edge complex plays a role in the Rac/actin/PIP3 positive feedback loop that modulates the linear cascades that connect chemoattractant receptors to the cytoskeleton in hematopoietic cells. Thus, Hem-1 and its associated protein complex appear to play an important role in organizing the leading edge of motile cells. According to this model, ARHGAP4's GAP activity would be strategically localized to terminate the Rac-mediated actin polymerization at the leading edge of migrating cells. In addition, the increased axon outgrowth and/or cell migration produced by expression of dominant-negative (R562A) ARHGAP4 or by application of siRNA suggests that ARHGAP4 keeps these complexes inactive, presumably until activated by a chemoattractant signal. The chemoattractant signal could either inactivate ARHGAP4 or lead to its dissociation from the complex. Although this model does not preclude the possibility that ARHGAP4 also could be activated by direct binding to chemorepulsive receptors in a manner similar to the activation of slit-Robo GAP by Robo receptors (Peck et al. (2002) FEBS Lett 528:27-34; Wong et al. (2001) Cell 107:209-221), there currently is no evidence to support this model. Unlike its restricted distribution in postnatal brain, ARHGAP4 is widely distributed in the CNS during embryonic development. This widespread distribution suggests that ARHGAP4 may play a central role in the regulation of cell and axon motility during embryonic development. For example, ARHGAP4 could be the inhibitory counterpart to p190GAP. Mice with p190 RhoGAP mutations exhibit defects in axon guidance and fasciculation (Brouns et al. (2001) Nat Cell Biol 3:361-367). In contrast to ARHGAP4, p190 RhoGAP overexpression leads to an increase in neurite outgrowth. Thus, proper neuronal development may require balanced regulation of ARHGAP4 and other GAPs. Reduction or inhibition of endogenous ARHGAP4 function using siRNA or by expression of the R562A dominant negative mutant protein enhanced cell migration and axon outgrowth, suggesting that the inhibition of motility involves the activation of ARHGAP4. Thus, motile cells appear to regulate the rate of motility by removing some of the inhibitory or braking action mediated by ARHGAP4. These observations suggest that downregulation of ARHGAP4 function could have a significant impact on the repair of CNS injury and may provide an important therapeutic strategy for enhancing recovery from CNS injury or neurodegenerative diseases. In summary, we have examined the structure and function of ARHGAP4, a novel RhoGAP molecule. Our results show that ARHGAP4 is a complex molecule whose N-terminus, GAP and C-terminus play different but essential roles in this protein's inhibition of cell and axon motility. The N-terminal amino acids 1-71 are important for the localization of ARHGAP4 to the cell periphery and axon growth cone, which appears be essential for its inhibition of cell motility. The C-terminus also appears to mediate protein-protein interactions. Together, these observations suggest that the function of ARHGAP4 depends on its interactions with protein complexes located in the periphery of motile cells and axon growth cones. Thus, ARHGAP4 may be important not only for understanding neuronal development, but also for the repair of CNS damage following traumatic injury or disease.

### Experimental Methods

### Dentate Explant Cultures

Explant cultures of the dentate gyms were prepared from 4-day-old Sprague Dawley rat pups. Rats were decapitated, and 200 μm transverse hippocampal slices were prepared using a WPI Vibroslice. A cut was made along an axis perpendicular to the pyramidal cell layer and in front of the open end of the granule cell layer using a #15 scalpel, and the dentate was carefully removed from the hippocampus. The dentates were further processed to remove CA3c pyramidal cells and much of the hilus. Thus, dentate explants consisted almost entirely of the dentate granule cell layer and the molecular layers.

Dentate explants were placed on 25 mm Nunc Anopore Membranes coated with polylysine (Sigma, St. Louis, Mo.) and laminin (Invitrogen, Carlsbad, Calif.) and maintained in 6 well tissue culture plates containing 1.5 ml Neurobasal/B27 medium (Invitrogen).

### Dissociated Dentate Cultures

The dentate gyms was microdissected from 200 μm hippocampal slices prepared from postnatal day 5-7 rats as previously described (Butler et al., 2004). The dentates were rinsed once in Neurobasal/B27 and then dissociated in Neurobasal/B27 with papain (2 μg/ml at 15-23 U/μg) for 30 min at RT. After incubation, dentates were rinsed and triturated gently 3 times, and

recovered material was centrifuged at 1000×g for 10 min. The pellet was resuspended in Neurobasal/B27 and seeded at a concentration of about 50-100 cells/mm2 onto polylysine/laminin coated coverslips. Neurons were transfected at 3 DIV and analyzed at 5 DIV.

### NIH/3T3 Cell Culture

NIH/3T3 cells were obtained from the American Type Culture Collection (ATCC, cell line CRL-1658) and were cultured according to ATCC protocol using Dulbecco's Modified Eagle's medium plus 10% bovine serum (DMEM+10% bovine serum). Construction of ARHGAP4 cDNA expression vectors ARHGAP4cDNA was generated by polymerase chain reaction (PCR) using the pεMTH-ARHGAP4 plasmid as a template (Foletta et al., 2002). Specific primers (5′-end primer: 5′CTAGGCGGCCGCATGGCGGCGCACGGGA AGTTGCGG-3′ (SEQ ID NO: 5); 3′-end primer: 5′-GCTAGAATTCGACTGGCTTGCGAGTTGAATCTGG-3′(SEQ ID NO: 6)) were used to insert NotI and EcoRI restriction sites into the 5′ and 3′ ends, respectively, of the ARHGAP4 cDNA. The NotI/EcoRI fragment was then subcloned into the multiple cloning site of the pCMV-Tag 4A vector plasmid (Stratagene, La Jolla, Calif.) to drive expression of a full-length ARHGAP4 (encoding amino acids 1-965) fusion protein with a carboxyterminal FLAG epitope: (1-965) FLAG. To generate the vector encoding a truncated ARHGAP4 fusion protein (72-965)-FLAG, specific PCR primers (5′-end primer: 5′ CATAGCGGCCGCATGGAA CGCTTTACTAG-3′ (SEQ ID NO: 7); 3′-end primer: 5′GCTAGAATTCGACTGGCTTGCGAGTTG AATCTGG-3′ (SEQ ID NO: 8)) were used to generate NotI/EcoRI fragments that were then subcloned into pCMV-Tag 4A, as above. To generate the vector encoding a truncated ARHGAP4 fusion protein (1-770)-FLAG, specific PCR primers (5′-end primer: 5′-CTAGGCGGCCGCATGG CGGCGCACGGGAAGTTGCGG-3′ (SEQ ID NO: 9); 3′-end primer: 5′-GCTAGAATTCAG CCTCCACAACTCCCTCG-3′ (SEQ ID NO: 10)) were used to generate NotI/EcoRI fragments that were then subcloned into pCMV-Tag 4A, as above. To generate the vector encoding a full-length ARHGAP4-carboxy terminal EYFP fusion protein (1-965)-EYFP, PCR primers (5′-end primer: 5′-AGAAAGATCTATGGCGGCGCACGGG-3′ (SEQ ID NO: 11); 3′-end primer: 5′-CTAGAATTCCGA CTGGCTTGCGAGTTGAATCTG-3′ (SEQ ID NO: 12)) were used to introduce BglII and EcoRI restriction sites into the ARHGAP4 cDNA for subcloning into the pEYFP-N1 vector (Clontech, Mountainview, Calif.). The same strategy was used to generate a vector encoding an ARHGAP4 fragment as the fusion protein (1-71)-EYFP, using PCR primers (5′-end primer: 5′-AGAAAGATCTA TGGCGGCGCACGGG-3′ (SEQ ID NO: 13); 3′-end primer: 5′ GGACGAATTC AGTATGTTCCAGCAGCAT-3′ (SEQ ID NO: 14)). The ARHGAP4 vector encoding (1-965)-FLAG was used as a template for site-directed mutagenesis to create cDNA that encoded an arginine→alanine substitution at amino acid 562 (R562A) in the full-length protein. The Stratagene QuikChange Multi Site-Directed Mutagenesis Kit (Stratagene #200514) was used according to the manufacturer's protocol, and mutant cDNA was generated using the 5′-phosphorylated primer (5′-CTGCAACATGAAGGCATCTTCG CGGTATCAGGTGCCCAGG-3′ (SEQ ID NO: 15), base pair changes shown in bold). Reaction products were incubated with DpnI to digest parental cDNA templates and were then transformed into XL-10-Gold Ultracompetent bacteria. The vector encoding an ARHGAP4 fusion protein, (R562A)-EYFP, was generated using the same PCR primers that were used to make the vector encoding ARHGAP4 (1-965)-EYFP. All plasmids were purified using the Qiagen Maxi Plasmid Kit (Qiagen, Inc., Valencia, Calif.) and modifications were verified by sequencing. In transfected NIH/3T3 cells (see below), protein expression driven by these vectors resulted in immunoreactive proteins of the expected apparent molecular weights by Western Analysis (see below) with the anti-FLAG antibody described above.

ARHGAP4 Gene Silencing with Small Interfering RNA

Small interfering RNAs (siRNAs) were generated using the Silencer siRNA Construction Kit (Ambion, Inc.) (Chen et al. (2005) J. Biol Chem. 280:2700-2707). Nucleotides 203-223 of mouse ARHGAP4 mRNA were chosen as targets for silencing using the Ambion siRNA Target Finder program. Briefly, complementary DNA oligonucleotides containing this region of ARHGAP4 cDNA and a complementary region of the 3′ end of the T7 promoter were annealed to a T7 promoter primer and filled in with Klenow DNA polymerase to generate double-stranded templates. ARHGAP4 oligonucleotides: 5′-ACAAGTTGGCTGAACGCTTTACCTGTCTC-3′ (SEQ ID NO: 16), and 5′ TAAAGCGTT CAGCCAACTTGTCCTGTCTC-3′ (SEQ ID NO: 17). Control oligonucleotides: 5′ ACAAATTAGCGGAGCGA TTCACCTGTCTC-3′, and 5′TGAATCGCTCCGCTAATTTGTCCTGTCTC-3′ (SEQ ID NO: 18) (6 underlined nucleotides encode silent mutations). The resulting double-stranded DNA templates were used to generate siRNA strands in in vitro transcription reactions using T7 RNA polymerase. ARHGAP4 or Control reaction products were combined to permit annealing of the siRNA strands, and DNA template removal, single-stranded RNA removal, and purification, were done according to the manufacturer's protocol. siRNAs were transfected into NIH/3T3 cells using Lipofectamine 2000 at a final concentration of 2 nM. After transfection, cells were cultured an additional 2 days prior to Western analysis or immunohistochemistry for wound assays.

### Transfection Methods for Dentate Explants

The manufacturer's in vivo transfection protocol was used to transfect dentate explants using ExGen 500. Briefly, 20 μg of plasmid expression vectors were added to 50 μl of sterile 5% glucose and 3.6 μl ExGen 500 in vivo transfection reagent (#R0521, Fermentas, Hanover, Md.). The DNA was condensed for 10 min at room temperature with the ExGen 500. Then, 1 μl of the transfection solution was applied to each explant using a micropipette, and cultures were returned to the incubator for 48 h. These interface cultures are not submerged in medium, therefore transfection reagents stay in contact with cultures until they eventually become diluted by the growth medium located below the porous culture insert. Transfection reagent volumes were sufficient to cover entire explants.

### Transfection Methods for Dissociated Neurons and NIH/3T3 Cells

Lipofectamine 2000 (Invitrogen) was used to transfect NIH/3T3 cells and dissociated hippocampal neurons according to the manufacturers' protocols Immunostaining Immunostaining of NIH/3T3 cells and dissociated neurons was performed as previously described (Oreliana and Marfella-Scivittaro (2000) Pediart. Res. 54:406-412); Marfella-Scivittaro et al. (2002) Am. J. Physiol. 282:C693-C707; Oreliana et al. (2003) J. Biol. Chem. 275:21233-21240); (Butler et al., 2004). Cells or explants were incubated with 0.1% Triton X-100 for 1-5 min and then incubated in block (10% serum in PBS) for 30 min at room temperature, washed 3 times for 5 min each with phosphate-buffered saline (PBS), and incubated with the primary antibody diluted in block for 1 h at 37° C. Samples were washed 3 times for 5 min each with block, incubated with secondary antibody for 1 h at 37° C., and washed 3 times for 5 min each with PBS. Primary antibodies used in this study were anti-GFAP (1:200, MP Biomedicals, Irvine, Calif.; or 1:1000, Sigma, St. Louis, Mo.), rabbit polyclonal antibody anti-ARHGAP4 (1:1000; Foletta et al., 2002), anti-β-Tubulin III (1:1000, Sigma), M2 anti-FLAG (1:250-1:1000, Sigma), and JL8 anti-GFP (1:1000, BDClontech). The cell adhesion molecule L1 was identified using a rabbit polyclonal antibody (1:1000, gift from Dr. Vance. The polyclonal antibody against ARHGAP4 was generated by Foletta et al. using amino acids 4-20, which a BLASTsearch showed to be unique to ARHGAP4. The antibody was affinity purified using amino acids 4-20 (Foletta et al., Experimental methods), and the authors demonstrated that pres absorption of the antibody with this unique sequence of amino acids eliminated specific immunoreactivity in rat hippocampal sections (Foletta et al., FIG. 8B). Secondary antibodies used were obtained from Molecular Probes (Eugene, Oreg.), and are noted in the Figure legends. F-actin was detected using fluorescent phalloidin conjugates from Molecular Probes.

Photomicrographs were obtained using a Leica TCS confocal microscope or a SPOT Digital Camera attached to a Nikon Optiphot-2 Fluorescence Microscope. SDS-PAGE and Western analysis Equal quantities of experimental protein samples from cosedimentation assays and from tissue and cell homogenates were subjected to SDSPAGE, and transferred to a solid support for Western analysis and ECL (Amersham Biosciences, Piscataway, N.J.) signal detection, as described (Oreliana and Marfella-Scivittaro, 2000; Marfella-Scivittaro et al., 2002; Oreliana et al., 2003). To confirm equal sample loading, blots werestripped and reprobed for immunoreactive α-tubulin expression (anti-α-tubulin, 1:200, Molecular Probes/Invitrogen, Carlsbad, Calif.) or using the C2 anti-Actin antibody (1:500, Santa Cruz Biotechnology, Santa Cruz, Calif.). Densitometric quantitation of immunoreactive signals on exposed films was performed using Kodak 1D software (Eastman Kodak Co., Rochester, N.Y.).

### Axon and Astrocyte Outgrowth Assay in Dentate Explants

Mossy fiber axon and astrocyte outgrowth was analyzed as previously described by our lab (Butler et al., 2004). Briefly, the granule cell layer was dissected from P4 rat hippocampi, plated on laminin-coated tissue culture inserts, and transfected as described above. An almost pure population of MF axons emerges from the edges of these round explants and extends onto the laminin substrate. Axons were visualized using an anti-β-tubulin III antibody. Explant images were obtained using the SPOT Digital Camera attached to a Nikon Optiphot-2 Fluorescence Microscope. The area of axon outgrowth was measured using the software supplied with the SPOT CCD camera. Total area (axons+explant) was obtained by drawing a circle that touched the distal tips of the elongated axons emanating from around the explant. The area of axon outgrowth was obtained by subtracting the area of the DAPI-stained explant from the Total area. The area of axon outgrowth was then divided by the area of the explant to normalize for the size of each individual explant. All values were then normalized for outgrowth with the parent vector (FLAG). These area measurements represent the outgrowth of a large population of axons rather than the length of a single axon. Astrocytes were visualized using an anti-GFAP antibody, and outgrowth was measured as for axons. Axon length measurement in dissociated dentate cultures

The cells were transfected at 3 DIV and fixed at 5 DIV. Cultures were transfected with ARHGAP4-FLAG cDNA expression vectors and visualized using FLAG immunocytochemistry. Control cultures were transfected using the pEYFP-N1 vector (Clontech, Mountainview, Calif.) and visualized using native EYFP fluorescence. Measurements were conducted as described (Kawano et al. (2005) Mol. Cell. Biol. 25:9920-9935), and only neurons that were not in contact with other cells or processes were analyzed.

### NIH/3T3 Migration Assay

A typical wound assay was used to measure NIH/3T3 cell migration. Briefly, a scratch/wound was made in a confluent area of the culture plate and the rate of cell migration towards the center of the wound was measured. Transfected cells were assessed 2, 4 and 8 h after wounding. Migration of transfected cells was quantified by counting the number of EYFP-positive cells at the line that represents the leading edge of migrating cells in the wound (fastest cells), and dividing by the total number of EYFP-positive cells between the wound edges (shown in FIG. 7 Panel A). All percentage values were normalized to control values (cells expressing EYFP alone) and the ratio for control cells is equal to 1.0 (represented by the horizontal dashed line shown in FIG. 7 Panel B).

### Recombinant GST-Fusion Proteins

The GAP domains (amino acids 474-743) of wild type ARHGAP4 (GST-WT-GAP) and R562A (GST-R562A-GAP) were generated by PCR using (1-965)-FLAG and (R562A)-FLAG as templates. Primers 5′ GCAGAATTCCTGCAGGCCAAGCATGAAAAGCTCCAG (SEQ ID NO: 19), and 3′-CATAAGCTTCTAGCTCTCCAACTGGCCATCCCCCAG (SEQ ID NO: 20) introduced EcoRI and HindIII sites (in bold) to clone into the GST-fusion expression vector pET-41a (Novagen). Protein purification was performed as previously described (Tong et al. (2005)). Vectors encoding GST-WT-GAP and GST-R562A-GAP were transformed into ONE SHOT BL21(DE3) bacterial cells (Invitrogen) and screened according to the manufacturer's protocol. Positive colonies were inoculated in LB+50 μg/ml Kanamycin and grown overnight at 225 rpms, 37° C. Cultures were diluted 1:100 in fresh LB+Kanamycin, grown for 3 h, then induced with 0.1 mM IPTG and grown overnight at 225 rpms, 30° C. The cells were pelleted and resuspended in GST-binding buffer (50 mM phosphate buffer pH 6.8, 100 mM NaCl, 4 mM DTT, 4 mM MgCl2)+sigma protease inhibitor cocktail, then sonicated on ice. Insoluble debris was cleared by centrifugation and the supernatant fractions were filtered through a 0.22 μm filter. GST-BIND Resin (Novagen) was equilibrated in GST binding buffer then combined with the resulting lysates for 20 mM at RT. Lysates were removed by gravity filtration on a column and the beads were washed 3× in GST-binding buffer. GST-fusion proteins were eluted off the beads with (100 mM Tris pH 8.5, 20 mM glutathione), and verified by SDS-PAGE and coommassie staining.

### In Vitro GAP Assay

The RhoGAP Assay Biochem kit #BK105 (Cytoskeleton) was used to determine the relative GAP activity of both GST-WT-GAP and GSTR562A-GAP recombinant proteins corresponding to amino acids 474-743 of ARHGAP4. Briefly, GST-WT-GAP, GST-R562A-GAP, RhoA-His, Rac1-His, and Cdc42-His proteins were diluted to 50 μM in nanopure water at 4° C. Reactions were set up in triplicate to examine GST-WT-GAP or GST-R562A-GAP+RhoA-His, Rac1-His or Cdc42-His, as well as reactions with either GTPase alone or the GST-WT-GAP or GST-R562AGAP alone. Reactions were combined with 1× Reaction Buffer in a 96 well plate on ice, and GTP was added to each well at a final concentration of 200 μM. The plate was shaken at 200 rpm, 5 s. and then incubated at 37° C. for 20 min. At the end of the reaction, 120 μl of Cytophos reagent was added to each well. The reactions were incubated for 10 min. at RT and then the absorbance was read at 650 nm to assay the level of GTP hydrolysis. Reactions containing 1× Reaction buffer+Cytophos reagent only were used as background controls.

### Statistical Determinations

Statistical determinations were made using SigmaStat V3.01 software (Systat Inc., Point Richmond, Calif.).

### Example 2

### Decreasing ARHGAP4 Expression Using shRNA can Enhance Regeneration of Spinal Cord Axons In Vivo

Rationale for Lentiviral shRNA Vector:

Our data has shown that short interfering RNA (siRNA) treatment of NIH/3T3 cells reduced ARHGAP4 expression by 40% and enhanced cell motility (FIG. 8). siRNA is a short double-stranded RNA oligo (approximately 20mer) that can silence gene expression by leading to the degradation of RNA or the inhibition of translation (Hannon (2002) Nature 418:244-251). While siRNA is a useful experimental tool, its ability to knockdown protein expression can be limited by its transfection efficiency and/or finite life span. The use of shRNA lentiviral vectors has been an effective approach to generating the type of long-term knockdown of proteins that are necessary for gene therapy (Mitta et al. (2005) Metab Eng. 7:426-436; Zuffrey et al. (1998) J. Virol 72:9873-9880). The short-hairpin sequence links the 2 siRNA strands, and allows the double-stranded siRNA oligo to be generated using a viral expression vector. For these studies, the short-hairpin loop sequence (TTCAAGAGA (SEQ ID NO: 21)) described by Brummelkamp et al. will be incorporated between the siRNA sequences (Brummelkamp et al. (2002) Science 296:550-553). A lentiviral vector was chosen because it can infect non-cycling and post-mitotic cells and generate long-term expression of the transgene. While these viruses initially raised safety concerns, the latest vectors have been heavily modified to remove the virulent genes and to prevent viruses from replicating. These modifications include: 1) Packaging vector lacks both the 3′ long terminal repeats (LTRs) and has no viral packaging signal (Ψ); 2) the env, tat, rev, vpr, vpu, vif and nef viral genes have been deleted from the packaging vector; 3) Rev is supplied in trans on a different vector (RSV-Rev); 4) The vector expressing the packaged viral genome has a self-inactivating LTR (TATA box deletion) and expresses no viral gene products; 5) Envelope (VSVG) is expressed on a different vector. Thus, manufacture of the complete virus can only be generated in 293T cells that have been transfected with the plasmid containing the lentilox vector that includes the shRNA insert in addition to three packaging vectors (pMDLg/pRRE, CMV-VSVG and RSV-Rev). These self-inactivating lentivirus vectors are considered to be safe enough for clinical gene therapy (Mitta et al. (2005)).

We have achieved excellent results using a commercially available self-inactivating lentiviral vector (LentiLox 3.7, ATCC) to express shRNA against the mRNA binding protein hnRNP-U in hippocampal explant cultures that retain some of the organotypic structure of intact CNS tissue. This shRNA lentiviral vector produced a 73% knock down in the expression of hnRNP-U (FIG. 9). This vector also generates expression of EGFP using a separate CMV promoter, allowing us to identify the EGFP-labeled axons of cells expressing the shRNA. Lentivirus shRNA vectors have also been shown to produce long-term reduction of targeted proteins in the brains of intact animals (Sapru et al. (2006) Exp. Neur. 198:382-390; Bahi et al. (2005) J. Of Neurochem. 92:1243-1255), making these lentiviral vectors a useful tool for in vivo spinal cord regeneration experiments. We have designed an shRNA lentivirus vector against ARHGAP4 and a control shRNA using our existing siRNA sequence (see methods). If necessary, the Ambion siRNA Target Finder can be used to identify additional siRNA sequences and commercially available siRNA against ARHGAP4 are available from Ambion and other companies. Viral vectors will be made from all of these sequences and tested in vitro for their ability to knockdown expression of ARHGAP4. They will be quantitated using Western analysis as described in FIG. 8. Although human gene therapy trials are not planned for this proposal, future clinical trials can use the same strategy to generate shRNA against human ARHGAP4 (accession #NM 001666).

### Transection of the Dorsal Spinal Cord:

The dorsal spinal cord can be hemisected to cut the corticospinal tract as previously described. (Zheng et al. (2003) Neuron 38:213-224). Female 129 mice (6-14 weeks old) can be anesthetized with Avertin (1.3% tribromoethanol and 0.8% amyl alcohol; Sigma). The hair on the back can be shaved and swabbed with betadine before making a midline incision. The paravertebral muscles can be dissected from the vertebral column and a leminectomy will be performed at T7-T8. The dura can be punctured using a 30 gauge needle bilaterally on the lateral aspects of the cord. A 0.7 mm deep cut can be made through the dorsal cord to the level of the central canal using a 0.2 mm thick microknife (Fine Science Tools). The wounds on the back can be closed and the animal will be operated on a second time to stereotaxically inject lentivirus vector (to express GFP and shRNA against ARHGAP4) into the sensorimotor cortex. Thus, neurons and their axons in which ARHGAP4 expression has been knocked down can also be labeled with GFP, enabling us to identify regenerating corticospinal track axons in the spinal cord. Control animals can be injected with a lentiviral vector that expresses GFP only. Lentivirus injections will be made on the day of spinal cord hemisection Animals involved in experiments to knockdown expression of ARHGAP4 can be operated immediately to inject the lentiviral vector into sensorimotor cortical neurons. The animals can be placed in a Stoelting Stereotaxic Apparatus, and three 2 μl injections can be made per hemisphere at a rate of 0.5 μl/min using a 5 ml Hamilton with a 30 gauge needle (site one: 1 mm lateral, 0.5 mm anterior to bregma, 0.5 mm deep to the cortical surface; site two: 1 mm lateral, 0.5 mm posterior, 0.5 mm posterior to bregma, 0.5 mm deep to the cortical surface; site three 1 mm lateral, 1 mm posterior to bregma, 0.5 mm deep to the cortical surface). Following the completion of the surgeries, the animals can be placed on a heating pad until they recover from the anesthetic. They can receive daily antibiotic injections (Baytril) for two weeks following surgery to prevent urinary tract infections, which is the primary cause of death in these types of experiments. The animals can be treated for postoperative pain by giving them two applications (one every 24 hr) of rimadyl (Carprofen, 1 mg per 100 gm bw, s.c.; Pfizer). Urine can be expelled by pressing the abdomen twice daily for seven days and then once daily until bladder function is restored.

Sagittal sections of the cord can be analyzed at 400× magnification to quantify the number of intersections of GFP-labeled fibers with a dorsoventral line. Five −7 sagittal sections, including the section through the most midline portions of the main dorsal medial corticospinal tract and the 3 adjacent sections on either side of the midline are selected for each animal. Only those fibers running outside the main thick bundle of corticospinal tract fibers are counted, since these have been shown to represent regenerating fibers. To normalize for individual tracing variability, the number of GFP-labeled corticospinal tract fibers are determined by counting fibers at 1000× magnification in a transverse section of the medulla rostral to the pyramidal decussation. The number of labeled fibers at different distances from the lesion site can be averaged over the 5-7 sections and divided by the number of labeled fibers in the medulla.

To calculate the percent knockdown of ARHGAP4 in cortical motor neurons, a group of animals are injected with viral injection medium containing blue dextran dye to identify the region of injection. These animals are be fixed with paraformaldhyde so that this region can be carefully dissected and analyzed for ARHGAP4 expression using Western analysis. If necessary, the cortical region can be dissociated and the cell suspension enriched for the population of EGFP-expressing cells using Fluorescence Activated Cell Sorting. ARHGAP4 levels can be measured in this enriched population of GFP-expressing cells using Western analysis. ARHGAP4 levels in mice receiving injections of ARHGAP4 shRNA-expressing lentivirus vector are compared to levels in mice that received injections of control lentivirus vector.

### Anticipated Results:

We anticipate that when the lentivirus vector for expression of ARHGAP4 shRNA is injected into motor cortex, GFP labeled corticospinal tract axons will be visible in the dorsal lateral spinal cord. Some animals will be used to quantify the percent knockdown of ARHGAP4 expression in motor cortex using Western analysis, and we anticipate a 50-70% knockdown of protein expression in GFP-positive cells expressing shRNA against ARHGAP4. However, it is possible that we will underestimate the percent knockdown if our dissection of the cortical injection sites is too large and includes untransformed cells.

One of the big problems with SCI is that axons retract away from the site of injury. Our preliminary data that use explants with damaged axons demonstrated an increase in axon outgrowth within 48 hours following decrease in ARHGAP4 function (dominant-negative protein expression) or expression (siRNA experiments). Therefore, we anticipate that corticospinal tract axons will not exhibit their typical retraction following injury, but instead will move forward through or around the transacted region of the dorsal spinal cord. This may be a critical issue because when injured axons retract, reactive glial cells in the spinal cord have time to deposit inhibitory extracellular molecules such as Nogo and chondroitin sulfate proteoglycan, which help to form the inhibitory barrier against axon regeneration. Thus, in addition to ARHGAP4 inducing axon growth cones to increase their forward motility rather than retract, axons may be able to traverse the damaged site before reactive glia can deposit the maximum amount of inhibitory molecules. In the event that the transaction leaves a physical gap in the tissue, the increased motility of axons can enable them to navigate around the lesion.

### Decreasing ARHGAP4 Function by Expressing Dominant-Negative (DN) ARHGAP4 can Enhance Regeneration Spinal Cord Axons In Vivo.

Our preliminary data using hippocampal neurons in vitro showed that DN-ARHGAP4 (R562A ARHGAP4) is a potent enhancer of axon outgrowth (FIG. 6). The advantage of this approach is that DN-ARHGAP4 can be placed in the spinal cord at the site of injury, which eliminates the need to perform a second surgery to make injections viral vectors into the cortex. It also eliminates any safety concerns over the use of lentiviral vectors for clinical therapies. Lastly, the long-term expression of an exogenous gene product is not required, further adding to the safety of this therapeutic approach. The introduction of the DN-ARHGAP4 protein into cells will be accomplished using a commercially available product called CHARIOT (Active Motif). Chariot has been used in vitro and in vivo to rapidly (<1 minute) introduce antibodies and other large proteins into living cells (Coulpier et al. (2002) J. Biol Chem 277:1991-1999; Jurney et al. (2002) J. Neurosci. 22:6019-6028; Gallo et al. (2002) J. Cell Bio. 158:1219-1228; Morris et al. (2001) Nat. Biotech. 19:1173-1176).

Spinal cord transaction will be performed as described above. However, before closing the incision over the spinal cord, the DN-ARHGAP4/Chariot solution is applied directly into the lesion of the transected spinal cord. A dose-response curve is generated to determine the concentration of DN-ARHGAP4 needed to produce the best results. For these experiments, we can follow the manufacturer's guidelines and the reports in the literature. (Coulpier et al. (2002) J. Biol Chem 277:1991-1999; Jurney et al. (2002) J Neurosci. 22:6019-6028; Gallo et al. (2002) J. Cell Bio. 158:1219-1228; Morris et al. (2001) Nat Biotech. 19:1173-1176). 20 μl of Chariot can be mixed with 1-5 μg of protein, incubated 1 hour at room temperature. In addition, small injections of DN-ARHGAP4-Chariot solution can be made directly into the dorsal-lateral spinal cord on the cranial side of the transaction in order to maximize the concentration of DN-ARHGAP4 in the area surrounded the ends of the cut axons. GFP can be mixed with DN-ARHGAP4 to label the axons that take up the protein and to identify axons that regenerate across the lesion. Control animals can receive GFP alone.

### Anticipated Results

Although we anticipate that DN-ARHGAP4 will strongly enhance axon outgrowth, there is some concern that axons will simply sprout on the cranial side of the lesion rather than growing across the lesion. We assume that this type of sprouting would exacerbated by the presence of inhibitory molecules in and around the lesion site. This problem will be address by injecting chondroitinase ABC into the lesion along with DN-ARHGAP4 so that we induce an increased outgrowth together with a reduction in inhibition. However, the BioAxone results with Cethrin suggest that this might not be necessary. We do not believe that it will be necessary to develop a sustained release system to prolong the exposure and subsequent uptake of DN-ARHGAP4 in order to produce a maximal regenerative response. This is because Chariot has been shown to rapidly introduce available protein into cells. In addition, prolonged release of DN-ARHGAP4 may induce oversprouting (see for example FIG. 6A3).

We can also generate a cell-permeable form of DN-ARHGAP4 in the same way that BioAxone modified C3 protein to produce Cethrin. The modifications that were examined to generate cell permeable C3 fusion proteins were described (Winton et al. (2002) J. Biol Chem. 277:32820-32829). These modifications include the C-terminal addition of the HIV peptide Tat, the 3^{rd} helix of the fly protein Antennapedia homeodomain (Antp), addition of a proline-rich region and the addition of a seven amino acid arginine-rich region. Of these, the addition of a proline-rich region produced the most effective results. It is also possible that we will observe some additional positive effects of DN-ARHGAP4 as was reported for Cethrin, such as decrease cell death and a decrease in glial scar formation.

### Experimental Methods:

Viral shRNA Vector:

A viral vector will be generated to express shRNA to knock down expression of ARHGAP4 in cortical sensorimotor neurons and their axons in the spinal cord. We have previously used the LentiLox 3.7 [pLL3.7] lentivirus vector obtained from ATCC (VRMC-39) to express shRNA against the mRNA binding protein hnRNP-U. This is a self-inactivating lentivirus vector containing a CMV driven EGFP reporter and a U6 promoter upstream of cloning sites for shRNA expression (Rubinson et al. (2003) Nat Genet 33:401-406).

Ambion siRNA Target Finder programs was used to design functional siRNA sequence used to knock down ARHGAP4 expression (FIG. 8). This siRNA sequence will then be entered into the 2-shRNA Oligo Designer which will provide sequences for the sense and antisense oligos. The oligos will be annealed and then ligated with linearized LentiLox 3.7 vector by incubation at 14° C. for 16-24 h. The resulting plasmids will be amplified by transforming DH5a cells, purified using a Qiagen Maxiprep kit, and sequenced.

Lentivirus containing the shRNA vector will be generated by quadruple-transfecting pLL3.7 and three packaging vectors (pMDLg/pRRE, CMV-VSVG and RSV-Rev, Invitrogen) into 293T cells using Lipofectamine 2000 (Invitrogen) and collecting the resulting supernatant after 24 and 48 hours. The virus will be concentrated by ultracentrifugation (Beckman XL-100 Ultracentrifuge, SW 41 Ti head) at 95,000×g (23,500 rpm) for 90 min at 4° C. The supernatant will be removed and the virus pellet resuspended to a titer of approximately 10⁷ TU/ml, aliquoted, and stored at −80° C.

ARHGAP4 Gene Silencing with Small Interfering RNA:

Small interfering RNAs (siRNAs) were generated using the SILENCER siRNA Construction Kit (Ambion, Inc.) (Chen et al. (2005) J Biol. Chem. 280:2700-2702). Nucleotides 203-223 of mouse ARHGAP4 mRNA were chosen as targets for silencing using the Ambion siRNA Target Finder program. Briefly, complementary DNA oligonucleotides containing this region of ARHGAP4 cDNA and a complementary region of the 3′ end of the T7 promoter were annealed to a T7 promoter primer and filled in with Klenow DNA polymerase to generate double-stranded templates. ARHGAP4 oligonucleotides: 5′-ACAAGTTGGCTGAACGCTTTACCTGTCTC-3′ (SEQ ID NO: 22), and 5′-TAAAGCGTTCAGCCAACTTGTCCTGTCTC-3′ (SEQ ID NO: 23). Control oligonucleotides: 5′-ACAAATTAGCGGAGCGATTCACCTGTCTC-3′ (SEQ ID NO: 24), and 5′-TGAATCGCTCCGCTAATTTGTCCTGTCTC-3′ (SEQ ID NO: 25) (6 underlined nucleotides encode silent mutations). The resulting double-stranded DNA templates were used to generate siRNA strands in in vitro transcription reactions using T7 RNA polymerase. ARHGAP4 or Control reaction products were combined to permit annealing of the siRNA strands, and DNA template removal, single-stranded RNA removal, and purification, were done according to the manufacturer's protocol. siRNAs were transfected into NIH/3T3 cells using Lipofectamine 2000 at a final concentration of 2 nM. After transfection, cells were cultured an additional 2 days prior to Western analysis or immunohistochemistry for wound assays.

### Transfection Using ExGen 500:

Plasmid expression vectors encoding specific cDNAs will be prepared using the Qiagen Maxi Plasmid Kit (Qiagen, Inc., Valencia, Calif.). Twenty micrograms of the plasmid vectors will be added to 50 μl of sterile 5% glucose and 3.6 μl ExGen 500 in vivo transfection reagent (Fermentas R0521). The DNA will be condensed at room temperature with ExGen 500 for 10 minutes. The transfection solution will be loaded into glass micropipettes and pressure injected into the tissue.

1. A method of modulating cell growth and motility in a subject, the method including:
administering to a cell expressing a GTPase an amount of an ARHGAP4 regulating agent effective to regulate the growth and motility of the cell.

2. The method of claim 1, the cell expressing a GTPase comprising a nerve cell.

3. The method of claim 2, the nerve cell being located at a central nervous system or peripheral nervous system injury site.

4. The method of claim 3, the central nervous system injury comprising a spinal cord injury and the ARHGAP4 being administered directly to nerve cell at site of injury.

5. The method of claim 1, the ARHGAP4 regulating agent comprising a polypeptide or nucleic that inhibits the bioactivity or expression of ARHGAP4 in a cell.

6. The method of claim 5, the ARHGAP4 regulating agent comprising a competitive inhibitor of ARHGAP4.

7. The method of claim 6, the competitive inhibitor of ARHGAP4 including a GAP loss of function mutation.

8. The method of claim 7, the competitive inhibitor of claim 7 including the amino acid sequence of SEQ ID NO: 1.

9. The method of claim 1, the ARHGAP4 regulating agent comprising ARHGAP4, an ARHGAP4 mimetic, or a vector, which can be used to express ARHGAP4 in a nerve cell.

10-13. (canceled)

14. A method of promoting axonal growth in a nerve cell comprising;
administering to the nerve cell an ARHGAP4 regulating agent such that axonal growth occurs, wherein said agent regulates the expression or bioactivity of ARHGAP4 in the cell.

15. The method of claim 14, wherein regulation of the expression or bioactivity of ARHGAP4 in the cell comprises decreasing expression or bioactivity of ARHGAP4 in the cell.

16. The method of claim 15, the ARHGAP4 regulating agent comprising a competitive inhibitor of ARHGAP4.

17. The method of claim 16, the competitive inhibitor including a GAP loss of function mutation.

18. The method of claim 17, the competitive inhibitor having an amino acid sequence of SEQ ID NO:1.

19. The method of claim 14 further comprising administering to the cell an additional agent that reduces inhibition of axon outgrowth.

20. The method of claim 14, the ARHGAP4 regulating agent comprising an interfering RNA that inhibits expression of ARGAP 4 in the cell.

21-26. (canceled)

27. A method of regulating cell motility, comprising:
administering to the cell a therapeutically effective amount of an ARHGAP4 regulating agent, wherein said agent regulates the bioactivity or expression of ARHGAP4 in the cell.

28. The method of claim 27, wherein the ARHGAP4 regulating agent decreases cell motility.

29. The method of claim 28, the cell comprising a metastatic neoplastic cell and the ARHGAP4 regulating agent decreasing neoplastic cell motility.

30. The method of claim 27, wherein the cell is a fibroblast cell and the ARHGAP4 regulating agent increases cell motility of the fibroblast cell.

31. The method of claim 30, the ARHGAP4 being administered to a fibroblast cell at the site of a wound to promote fibroblast motility at the wound.
